(19) United States
(12) Patent Application Publication
(10) Pub. No.: US 2025/0351857 A1
(43) Pub. Date: Nov. 20, 2025

(54) FOOD COMPOSITION AND METHOD FOR PRODUCING FOOD COMPOSITION

(71) Applicant: YUKIGUNI FACTORY CO., LTD., Minamiuonuma-shi (JP)

(72) Inventors: Masaharu KATO, Minamiuonuma-shi (JP); Masatoshi KERA, Minamiuonuma-shi (JP); Mizuho KIWADA, Minamiuonuma-shi (JP); Keiichiro KANDA, Minamiuonuma-shi (JP)

(21) Appl. No.: 19/288,959

(22) Filed: Aug. 1, 2025

Related U.S. Application Data
(63) Continuation of application No. PCT/JP2024/003594, filed on Feb. 2, 2024.

(30) Foreign Application Priority Data
Feb. 3, 2023 (JP) 2023-015661

(51) Int. Cl. A23L 31/00; A23J 3/14; A23L 29/244; A23L 29/256; C12N 9/10

(52) U.S. Cl. CPC A23L 31/00; A23J 3/14; A23L 29/244; A23L 29/256; C12N 9/1044; C12Y 203/02013

(57) ABSTRACT

It is possible to provide a food composition obtained using mushrooms as a raw material and capable of exhibiting a texture similar to that of meat. One aspect of the present invention is a food composition. The food composition includes: mushroom-derived solid content; and a structuring material that structures the solid content together. The solid content in the food composition is 1.3 to 49.0 mass %, and the content of the structuring material in the food composition is 1.5 to 90.0 mass %. In addition, the food composition has a peak at a strain of 10% to 60% and a load of 2 N or more at a strain of 30% in the following load test. (Load test) A cylindrical plunger having a diameter of 1 cm is attached to a rheometer, and the load of a test sample (height: 3 cm) is measured at a speed of 1 mm/s at room temperature.

### TECHNICAL FIELD

The present invention relates to a food composition and a method for producing a food composition.

### BACKGROUND ART

Among fungi, those forming relatively large fruiting bodies are regarded as mushrooms and belong mainly to the phylum Basidiomycota, with some included in Ascomycota.

In Non Patent Literature 1, 4000 kinds of mushrooms grow in Japan, and about 400 kinds of mushrooms are used for food or medicine.

Mushrooms not only have deliciousness but also contain unique dietary fiber and vitamins, and various health effects thereof have been reported. For example, Non Patent Literature 2 reports that mushrooms have anti-obesity, anti-diabetic, anti-cancer, immunomodulatory, anti-inflammatory effects, and the like.

According to Non Patent Literature 3, it is reported that the number of Japanese patients with lifestyle-related diseases has reached 18.5 million, which is about 15% of the total population, and 2 out of 3 Japanese people die from any of “cancer, heart disease, and cerebrovascular disease,” which are lifestyle-related diseases. A lifestyle-related disease is said to develop and progress due to accumulation of daily lifestyle habits such as unbalanced diet, lack of exercise, and stress, and in particular, excessive intake of energy and salt is thought to be significantly involved. On the other hand, in addition to the health effects described above, edible mushrooms are also considered to be effective for preventing lifestyle-related diseases because they are foods containing low amounts of lipids, carbohydrates, and calories and containing a significantly low level of sodium.

Furthermore, Non-Patent Literature 4 reports that β-glucan, a type of dietary fiber, exhibits different health effects depending on whether it is derived from plants or from fungi such as mushrooms, and that immunomodulatory and disease-preventive effects are stronger in β-glucan derived from fungi. Non Patent Literature 5 reports that, among β-glucans contained in mushrooms, poorly soluble β-glucan particularly exhibits strong immunomodulatory activity.

Mushrooms tend to be traditionally consumed more often in autumn and winter, as they are generally harvested in large quantities in the wild during autumn. In addition, due to such traditions, their culinary use has also been limited to dishes such as hot pot dishes, stewed dishes, and soups. Furthermore, the use of mushroom-derived poorly soluble β-glucan, which is expected to have strong immunomodulatory and disease-preventive effects, in processed foods is rarely seen.

On the other hand, in recent years, it has become possible to artificially cultivate mushrooms and to consume them throughout the year. However, due to the traditional limitation of eating scenes, consumption is still higher in autumn and winter, and it cannot be said that many consumers enjoy its deliciousness or health value over a year.

Patent Literature 1 has filed an application for use of a special facility to create a specific atmospheric environment and cultivate mycelia, rather than fruiting bodies, to a large size for edible use.

Patent Literature 2 has filed an application for producing mycelial bodies rich in protein from used food materials for edible use.

Patent Literature 3 discloses a meat product containing stir-fried mushroom pieces and a binding component, and describes its use as an alternative to livestock meat, of which there is a concern of insufficiency thereof in the future.

### CITATION LIST

### Non Patent Literature

Non Patent Literature 1: Science of mushrooms, Japan, 1997
Non Patent Literature 2: Foods 2016, 5, 80; doi: 10.3390/foods5040080
Non Patent Literature 3: Ministry of Health, Labour and Welfare “Overview of Patient Survey in 2017”
Non Patent Literature 4: J. Fungi 2020, 6, 356; doi: 10.3390/jof6040356 Non Patent Literature 5: Moleculers 2015, 20:9745-9766

### Patent Literature

Patent Literature 1: WO2021/092051 A1
Patent Literature 2: WO2022/136708 A
Patent Literature 3: Japanese Patent No. 6885699

### SUMMARY OF INVENTION

### Technical Problem

Several items of prior art have been recognized for processed foods containing mushrooms. However, all of them are merely described within the scope of conventional common technical knowledge, and the meat-like texture and flavor preferred by many consumers are not realized, and there is room for improvement.

The present invention has been made in view of the above problem, and an object of the present invention is to provide a technique relating to a food composition obtained using mushrooms as a raw material and capable of exhibiting a texture similar to that of meat.

### Solution to Problem

One aspect of the present invention is a food composition. The food composition is a food composition including: mushroom-derived solid content; and a structuring material that structures the solid content together, in which the solid content in the food composition is 1.3 to 49.0 mass %, the content of the structuring material in the food composition is 1.5 to 90.0 mass %, and the food composition has a peak at a strain of 10% to 60% and a load of 2 N or more at a strain of 30% in the following load test.

(Load test)

A cylindrical plunger having a diameter of 1 cm is attached to a rheometer, and the load of a test sample (thickness: 3 cm) is measured at a speed of 1 mm/s at room temperature.

The food composition according to the above aspect may contain 0.4 to 25.0 mass % of dietary fiber. The food composition may contain 0.05 to 8.0 mass % of mushroom-derived poorly soluble β-glucan.

In the food composition according to the above aspect, the structuring material may be selected from the group consisting of non-mushroom-derived protein materials, protein crosslinking enzymes, alginic acids, and mannans. In this case, the non-mushroom-derived protein materials may include one or more selected from the group consisting of beans, cereals, natural meats such as livestock meat, and dairy products. The protein crosslinking enzymes may include transglutaminase. The alginic acids may be selected from the group consisting of alginic acid, sodium alginate, potassium alginate, calcium alginate, alginate esters, and ammonium alginate. The mannans may be selected from the group consisting of konjac powder, konjac mannan, and glucomannan.

In the food composition according to the above aspect, the mushrooms may be fruiting bodies.

In the food composition according to the above aspect, the mushrooms may be one or more selected from the group consisting of enokitake (Flammulina velutipes), eringi (Pleurotus eryngii), wood ear mushroom (Auricularia auricula-judae), shiitake (Lentinula edodes), button mushroom (Agaricus bisporus), nameko (Pholiota nameko), oyster mushroom (Pleurotus ostreatus), buna-shimeji (Hypsizygus marmoreus), and maitake (Grifola frondosa).

Another aspect of the present invention is a dry food composition. The dry food composition is obtained by drying the food composition according to any one of the above aspects.

Still another aspect of the present invention is a method for producing a food composition. The production method includes: a mixing step of mixing mushroom-derived solid content with a structuring material that structures the solid content together; and a standing step of allowing the mixture obtained by the mixing step to stand under an environment of 3° C. to 60° C. for 1.0 to 48 hours, in which the solid content in the food composition is 1.3 to 49.0 mass %, and the content of the structuring material in the food composition is 1.5 to 90.0 mass %.

In the method for producing a food composition according to the above-described aspect, the food composition may contain 0.4 to 25.0 mass % of dietary fiber. The food composition may contain 0.05 to 8.0 mass % of mushroom-derived poorly soluble β-glucan. The structuring material may be selected from the group consisting of non-mushroom-derived protein materials, protein crosslinking enzymes, alginic acids, and mannans.

### Advantageous Effects of Invention

According to the present invention, it is possible to provide a technique relating to a food composition obtained using mushrooms as a raw material and capable of exhibiting a texture similar to that of meat.

### BRIEF DESCRIPTION OF DRAWINGS

FIG. 1 is a graph showing results of a load test for each food composition of Examples 1 to 3.

FIG. 2 is a graph showing results of a load test for each food composition of Example 4 and Reference Example 1.

FIG. 3 is a graph showing results of a load test for each food composition of Example 5 and Comparative Example 3.

FIG. 4 is a graph showing a result of a load test for a food composition of Example 6.

FIG. 5 is a graph showing a result of a load test for a food composition of Example 7.

FIG. 6 is a graph showing results of a load test for each food composition of Examples 8 and 9.

FIG. 7 is a graph showing results of a load test for each food composition of Examples 10 and 11 and Comparative Example 4.

FIG. 8 is a graph showing results of a load test for each food composition of Examples 12 to 15 and Comparative Example 5.

FIG. 9 is a graph showing results of a load test for each food composition of Example 16 and Comparative Examples 7 and 8.

FIG. 10 is a graph showing results of a load test for each food composition of Examples 17 to 19.

FIG. 11 is a graph showing results of a load test for each food composition of Examples 20 to 23 and Comparative Example 9.

FIG. 12A is a graph showing results of a load test for each food composition of Examples 24 to 26.

FIG. 12B is a graph showing results of a load test for each food composition of Examples 27 to 29.

FIG. 12C is a graph showing results of a load test for each food composition of Examples 30 to 32.

FIG. 13 is a graph showing results of a load test for each food composition of Examples 33 and 34.

FIG. 14 is a graph showing results of a load test for each food composition of Example 35 and Comparative Examples 10 and 11.

FIG. 15 is a graph showing results of a load test for each food composition of Examples 36 to 39.

FIG. 16 is a graph showing results of a load test for each food composition of Comparative Examples 12 and 13.

FIG. 17 is a graph showing results of a load test for each food composition of Examples 40 and 41.

FIG. 18 is a graph showing results of a load test for each food composition of Examples 42 and 43.

FIG. 19 is a graph showing a result of a load test for a food composition of Example 44.

FIG. 20 is a graph showing results of a load test for each food composition of Example 45 and Comparative Example 14.

FIG. 21 is a graph showing results of a load test for food compositions of Examples 46 to 49.

FIG. 22 is a graph showing results of a load test for food compositions of Examples 50 to 53.

FIG. 23 is a graph showing results of a load test for food compositions of Examples 54 to 57 and Comparative Example 15.

### DESCRIPTION OF EMBODIMENTS

Hereinafter, embodiments of the present invention will be described in detail. In the present specification, the notation “a to b” in the description of a numerical range represents a or more and b or less, unless otherwise specified.

### (Food Composition)

The food composition according to an embodiment includes: mushroom-derived solid content; and a structuring material that structures the solid content together.

The mushrooms as a raw material of the solid content used in the food composition of the present embodiment are not particularly limited as long as they are edible, but are preferably one or more selected from the group consisting of enokitake (Flammulina velutipes), eringi (Pleurotus eryngii), wood ear mushroom (Auricularia auricula-judae), shiitake (Lentinula edodes), button mushroom (Agaricus bisporus), nameko (Pholiota nameko), oyster mushroom (Pleurotus ostreatus), buna-shimeji (Hypsizygus marmoreus), and maitake (Grifola frondosa) from the viewpoint of improving texture and being rich in nutrients.

The above mushrooms may be either mycelia or fruiting bodies, but are preferably fruiting bodies. By using the mushrooms of the fruiting bodies, the texture can be made more similar to that of meat.

The mushroom-derived solid content refers to a solid when mushrooms are dried and moisture is removed. For example, since maitake contains 93% of moisture, the mushroom-derived solid content of maitake itself is 7%. In other words, when the food composition is in a solid form, it refers to a component obtained by removing moisture derived from mushrooms from the solid content in the food composition, and when the food composition is in a liquid or fluid form, it refers to a component derived from mushrooms obtained by removing moisture in the food composition.

The mushroom-derived solid content in the food composition is preferably 1.3 to 49.0 mass %, more preferably 2.0 to 40.0 mass %, and still more preferably 3.0 to 30.0 mass %. By setting the mushroom-derived solid content within the above ranges, the texture of the resulting food composition can be made more similar to that of meat.

The mushrooms used may be in their raw state or in the form of processed mushroom products. Examples of treatments to obtain processed mushrooms include drying, heating, compression, and denaturation. In addition, the mushrooms to be used may include a dried product of an extract obtained from mushrooms.

The shape and size of the mushrooms to be used may be adjusted by grinding, cutting, or the like. From the viewpoint of improving texture, the particle diameter of the mushrooms during the grinding is, for example, preferably 0.5 mm or more and 10 mm or less, more preferably 1 mm or more and 8 mm or less, still more preferably 3 mm or more and 6 mm or less.

The mushrooms used may be pressed or compressed.

The food composition preferably contains dietary fiber in an amount of 0.4 to 25.0 mass %, more preferably 0.6 to 20.0 mass %, and still more preferably 1.1 to 15.0 mass %. By setting the content of the dietary fiber in the food composition within the above range, it is possible to obtain a food composition that is gentle on the intestinal environment while making the texture similar to that of meat.

The dietary fiber preferably contains mushroom-derived poorly soluble β-glucan. According to this, it is possible to obtain immunomodulatory effects such as anti-infective or anti-allergic effects.

The food composition preferably contains mushroom-derived poorly soluble β-glucan in an amount of 0.05 to 8.0 mass %, more preferably 0.15 to 7.0 mass %, and still more preferably 0.30 to 5.0 mass %. By setting the content of the mushroom-derived poorly soluble β-glucan within the above range, immunomodulatory effects such as anti-infective or anti-allergic effects can be sufficiently obtained.

The structuring material is not particularly limited as long as it has a function of structuring the mushroom-derived solid content together (in other words, a function to form a meat-like structure), but examples thereof include non-mushroom-derived protein materials, protein crosslinking enzymes, alginic acids, and mannans from the viewpoint of improving the meat-like structuring property while making the texture of the resulting food composition similar to that of meat. The protein material refers to a material containing 10% or more protein on a dry weight basis.

The content of the structuring material in the food composition is preferably 1.5 to 90.0 mass %, more preferably 2.0 to 70.0 mass %, and still more preferably 3.0 to 55.0 mass %. By setting the content of the structuring material within the above ranges, the texture of the resulting food composition can be made more similar to that of meat.

Examples of the non-mushroom-derived protein materials include one or more non-mushroom-derived protein materials selected from the group consisting of beans, cereals, livestock meats, and dairy products. Examples of the bean-derived protein materials include soy protein, pea protein, defatted soybeans, and okara powder. Examples of the cereal-derived protein materials include oatmeal and oat bran. Examples of the dairy products include casein, milk protein, skim milk powder, and whole milk powder.

When the content of the non-mushroom-derived protein materials in the food composition is quantified, it can be calculated by measuring the total content of protein in the food composition by a measurement method such as a combustion method, and then subtracting the content of mushroom-derived protein contained in the food composition from the total content.

Specifically, the ratio of the amount of protein (Y g) per 100 g of raw mushrooms to the amount of poorly soluble β-glucan (X g) per 100 g of raw mushrooms is a value (Y/X) determined according to the type of mushroom as shown in Table 1 below. The content of mushroom-derived protein contained in the food composition can be calculated based on the content of poorly soluble β-glucan contained in the food composition and the above value (Y/X).

TABLE 1

| | Amount of poorly soluble β-glucan | Amount of protein | |
|---|---|---|---|
| | (X g) per 100 g of raw mushrooms | (Y g) per 100 g of raw mushrooms | Y/X |
| Bringi | 1.98 | 2.8 | 1.4 |
| Maitake | 1.64 | 2.0 | 1.2 |
| Dried eringi | 11.29 | 16.0 | 1.4 |
| Pressed eringi | 2.11 | 4.8 | 2.3 |
| Button mushroom | 0.12 | 2.9 | 24.2 |
| Buna-shimeji | 1.66 | 2.7 | 1.6 |
| Shiitake | 0.55 | 3.0 | 5.5 |
| Enokitake | 0.58 | 2.7 | 4.7 |
| Wood ear mushroom | 0.09 | 0.7 | 7.8 |

Examples of the protein crosslinking enzymes include transglutaminase.

Examples of the alginic acids include one or more selected from the group consisting of alginic acid, sodium alginate, potassium alginate, calcium alginate, alginate esters, and ammonium alginate.

Examples of the mannans include one or more selected from the group consisting of konjac powder, konjac mannan, and glucomannan.

In the following load test, the food composition according to the embodiment has a peak at a strain of 10% to 60% and a load at a strain of 30% is 2 N or more, preferably 4 N or more, and more preferably 6 N or more. The upper limit of the load at a strain of 30% is not particularly limited as long as an appropriate biting texture can be obtained, but is preferably 400 N or less, and more preferably 200 N. The expression “having a peak” means that values before and after a peak value (maximum value) are smaller than the peak value within a strain range of 10% to 60%.

### (Load Test)

A cylindrical plunger having a diameter of 1 cm is attached to a rheometer, and the load of a test sample (cylindrical shape with a height of 3 cm) is measured at a speed of 1 mm/s at room temperature.

When the result of the load test satisfies the above conditions, the texture of the resulting food composition can be made more similar to that of meat.

### (Additive)

The food composition according to the embodiment may contain various additives as long as the meat-like texture is not impaired. Examples of the additives include well-known preservatives, colorants, seasonings, emulsifiers, and flavorings. In addition, the additives include food ingredients such as various types of sauces such as hamburger steak sauce.

In addition, the food composition according to the embodiment may contain a reinforcing agent that enhances the meat-like structuring effect of the structuring materials such as protein crosslinking enzymes, alginic acids, and mannans.

Examples of the reinforcing agent include one or more substances selected from the group consisting of phosphoric acid, phosphates, pyrophosphoric acid, pyrophosphates, polyphosphoric acid, polyphosphates, ammonium salts, lactates, sulfates, carbonates, and hydroxide salts. Although not particularly limited, examples thereof include trisodium phosphate, tetrasodium pyrophosphate, sodium polyphosphate, ammonium chloride, calcium lactate, calcium sulfate, sodium pyrophosphate, and sodium carbonate.

In addition, it is desirable that the above-described structuring by the structuring materials be performed with appropriate solid and moisture content. Therefore, as an auxiliary agent for enhancing the function of the structuring material by moisture adjustment, a so-called binding material may be included from the viewpoints of moisture adjustment, texture, water separation prevention, flavor, economic considerations, and the like. The binding material is not particularly limited, and examples thereof include eggs, starches, and breadcrumbs.

In addition, the food composition according to the embodiment may contain a polysaccharide from the viewpoint of maintaining the solidified state. Examples of the polysaccharide include one or more polysaccharides selected from the group consisting of curdlan, glucomannan, konjac powder, guar gum, carrageenan, gellan gum, xanthan gum, and tamarind gum. The polysaccharide may be referred to as a thickener or a thickening polysaccharide.

According to the food composition described above, it is possible to obtain a meat-like texture while using mushrooms as a raw material.

### (Use and Cooking Method of Food Composition)

The food composition according to the above-described embodiment can be served as hamburger steaks, croquettes, minced meat cutlets, meat dumplings, meatballs, sweet-and-sour pork, meat sauce, and the like by heating and cooking using a frying pan or a microwave oven. Various seasonings such as salt and soy sauce may be appropriately added to the food composition before or after heating and cooking.

### (Shape and Form of Food Composition)

The shape and form of the food composition according to the above-described embodiment can be a shape and form according to the purpose of use, and examples thereof include amorphous forms and pellet forms molded into a plate shape, a dice shape, a cylindrical shape, or the like.

### (Dry Food Composition)

The dry food composition according to an embodiment is obtained by drying the food composition of the embodiment described above. The dry food composition can be used as, for example, a food ingredient for jerky or a delicacy material. In addition, the dry food composition may be rehydrated with water and then used as a food ingredient.

### (Method for Producing Food Composition)

The method for producing food composition according to the embodiment includes: a mixing step of mixing mushroom-derived solid content with a structuring material that structures the solid content together; and a standing step of allowing the mixture obtained by the mixing step to stand under an environment of 3° C. to 60° C. for 1 to 48 hours. The mushrooms and the structuring material used are as described above. The standing time may be 2 hours or more, 4 hours or more, or 8 hours or more.

By the mixing step, the raw materials are mixed so as to be uniform. The formulating amount of the mushroom-derived solid content and the formulating amount of the structuring material in the mixing step may be adjusted so that the solid content is 1.3 to 49.0 mass % and the content of the structuring material is 1.5 to 90.0 mass % in the resulting food composition.

A step of grinding the mushrooms may be provided before the mixing step. By grinding the mushrooms, the texture of the resulting food composition can be made similar to that of meat. The particle diameter of the mushrooms during the grinding is preferably 0.5 mm or more and 10 mm or less, more preferably 1 mm or more and 8 mm or less, still more preferably 3 mm or more and 6 mm or less.

In addition, the mushroom-derived solid content may be heated in advance, for example, at 80° C. to 150° C. for 10 to 30 minutes. In the case of heating the mushroom-derived solid content, steaming, steam heating, boiling, or microwave heating is preferable, and heating such as stir-frying with oil may lower the efficiency of the subsequent structuring reaction, and therefore heating without using oil is preferable.

After the mixing step, the resulting mixture may be molded using a mold (for example, a cylindrical shape having a diameter of 4 cm and a height of 3 cm).

In the method for producing a food composition according to the embodiment, the structuring of the mushroom-derived solid content together can be sufficiently advanced by undergoing the standing step under the conditions described above, thereby enabling the production of a food composition having a peak at a strain of 10% to 60% and a load of 2 N or more at a strain of 30% in the above-described load test. In other words, according to the method for producing a food composition according to the embodiment, it is possible to obtain a food composition that provides a meat-like texture while using mushrooms as a raw material. By setting the temperature condition in the standing step to 3° C. or higher, it is possible to advance the structuring without freezing the food composition. In addition, by setting the temperature condition in the standing step to 60° C. or lower, the structuring of the food composition can be advanced without thermal deterioration.

The mushroom-derived solid content preferably contains 0.4 to 25.0 mass % of dietary fiber. The dietary fiber preferably contains mushroom-derived poorly soluble β-glucan. The structuring material is preferably selected from the group consisting of non-mushroom-derived protein materials, protein crosslinking enzymes, alginic acids, and mannans.

Although the embodiments of the present invention have been described above, these are merely examples of the present invention, and various configurations other than the above can be adopted.

### EXAMPLES

Hereinafter, the present invention will be described with reference to examples and comparative examples, but the present invention is not limited thereto.

Each food composition of Examples 1 to 3 and Comparative Examples 1 and 2 using mushrooms as a raw material was prepared according to the formulation shown in Table 2.

As mushrooms, eringi was used. Eringi was ground to a particle size of 3 mm or less using a food processor (MK-K81-W, Panasonic Corporation), and the ground eringi was heated in a pot with a lid for about 10 minutes.

The raw materials were mixed well so as to be uniform, molded using a cylindrical mold having a diameter of 4 cm and a height of 3 cm, and allowed to stand in a refrigerator set at 10° C. or lower for 20 hours.

“Activa TG-B Powder Coating” available from Ajinomoto Co., Inc. containing 0.5% transglutaminase, 2.5% sodium polyphosphate, 2.5% (anhydrous) sodium pyrophosphate, 2.0% silicon dioxide, and 92.5% milk protein was used as a transglutaminase preparation A, and “Kona no Sato Dayori Cornstarch” manufactured by Hinokuni Shokuhin Kogyo K.K. was used as starch.

In order to stop the structuring reaction, steaming was performed for 15 minutes in a covered pot to which a small amount of water had been added. The moldability was confirmed according to the “Method for confirming moldability” described below. Those that could not be molded before being left standing were deemed non-conforming. Dietary fiber was measured according to “Method for measuring dietary fiber.” In addition, the content of poorly soluble β-glucan was measured according to “Method for measuring fungi-derived poorly soluble β-glucan.” Sensory evaluation of each resulting food composition was performed according to the “Sensory evaluation method” described below.

TABLE 2

| | Structuring | Comparative | Comparative | | | |
|---|---|---|---|---|---|---|
| | material | Example 1 | Example 2 | Example 1 | Example 2 | Example 3 |
| Eringi | — | 0.0 | 10.0 | 20.0 | 30.0 | 50.0 |
| Transglutaminase preparation A | ◯ (93%) | 10.0 | 10.0 | 10.0 | 10.0 | 10.0 |
| Water | — | 45.0 | 36.0 | 27.0 | 18.0 | 0.0 |
| Starch | — | 45.0 | 44.0 | 43.0 | 42.0 | 40.0 |

| | | | | | |
|---|---|---|---|---|---|
| Total | 100.0 | 100.0 | 100.0 | 100.0 | 100.0 |
| Mushroom solid content | 0.0 | 1.0 | 2.0 | 3.0 | 5.0 |
| Dietary fiber | 0.0 | 0.3 | 0.7 | 1.0 | 1.7 |
| Poorly soluble β-glucan | 0.00 | 0.20 | 0.40 | 0.59 | 0.99 |
| Structuring material | 9.3 | 9.3 | 9.3 | 9.3 | 9.3 |

(Unit of numerical values: mass %)

As shown in Table 2, in Examples 1 to 3 and Comparative Example 2, 90% of the (raw) eringi used as a material was moisture, and the remaining 10% was solid content.

### (Method for Measuring Dietary Fiber)

Dietary fiber was determined by the sum of water-soluble dietary fiber and insoluble dietary fiber using a modified Prosky method. That is, 1 g of each dried powder sample was accurately weighed out, and each enzymatic degradation (amylase, protease, amyloglucosidase) treatment was performed. The enzymatically degraded product was subjected to suction filtration to obtain a filtrate and a residue.

The filtrate was subjected to ethanol precipitation to obtain a precipitate. The precipitate was washed with ethanol and dried, and then the amount of the dry substance and the ash content, which was a residue obtained by ashing by a direct ashing method, were measured. That is, the amount of water-soluble dietary fiber was determined from a value obtained by subtracting the ash content from the amount of the dry substance.

The residue was washed with ethanol and dried, and then the amount of the dry substance and the ash content were measured. That is, the amount of insoluble dietary fiber was determined from a value obtained by subtracting the ash content from the amount of the dry substance. In addition, the total amount of dietary fiber was determined by summing the amounts of water-soluble dietary fiber and insoluble dietary fiber obtained.

### (Method for Measuring Fungi-Derived Poorly Soluble β-Glucan)

Fungi-derived poorly soluble β-glucan is mainly β-1,3;1,6-glucan, which is different from β-1,3;1,4-glucan often found in plants, but no standardized analytical method is available. Therefore, the content was determined by combining the “total β-glucan measurement method,” for which an analysis method is available, and the “β-1,3;1,4-glucan measurement method,” for which an analysis method is likewise available.

That is, 5 times of distilled water was added to 20 g of each sample, then the mixture was well stirred, and extraction was performed at 121° C. using an autoclave. The extract was cooled at room temperature, and then centrifuged at 10,000 rpm using a centrifuge to obtain a poorly soluble fraction. The poorly soluble fraction was dried to obtain a powder. The extraction rate of the poorly soluble fraction was determined from the mass of the original sample and the mass of the poorly soluble fraction in powder form.

For the measurement of the total β-glucan content, a β-glucan assay kit (K-YBGL method) manufactured by Megazyme Ltd. was used. For the measurement of the β-1,3;1,4-glucan content, a β-glucan assay kit (K-BGLU method) manufactured by Megazyme Ltd. was used. The K-BGLU method is a measurement method that specifically degrades β-1,3;1,4-glucan using β-1,3;1,4-glucan-4-glucanohydrolase, and cannot measure β-1,3;1,6-glucan, which is mainly fungi-derived β-glucan. On the other hand, the K-YBGL method can determine both β-1,3;1,4-glucan and β-1,3;1,6-glucan as a combined value. Therefore, the fungi-derived poorly soluble β-glucan content was determined by subtracting the value of the K-BGLU method from the value of the K-YBGL method determined using the dry powder of the poorly soluble fraction, and multiplying by the extraction rate of the poorly soluble fraction described above. The results obtained regarding the content of poorly soluble β-glucan for each food composition are shown in Table 2.

### (Method for Confirming Moldability)

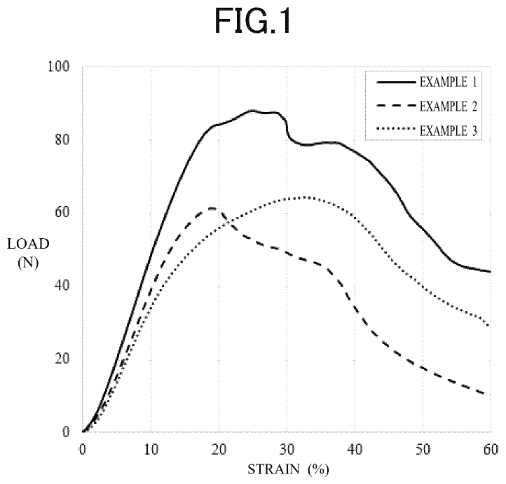

A cylindrical plunger having a diameter of 10 mm was attached to a rheometer (EZ-LX, Shimadzu Corporation), and a test force (load) was determined at a speed of 1.0 mm/s for a test product (height: 3 cm) at room temperature. From the obtained results, the presence or absence of a peak (corresponding to a breaking point) at a strain of 10% to 60% was evaluated, and a load (N) at a strain of 30% was obtained. The results on moldability obtained for each food composition are shown in Table 3. In addition, a graph showing results of a load test for each food composition of Examples 1 to 3 is shown in FIG. 1.

TABLE 3

| | Comparative | Comparative | | | |
|---|---|---|---|---|---|
| | Example 1 | Example 2 | Example 1 | Example 2 | Example 3 |
| Presence or absence of peak | Not moldable | Not moldable | Present | Present | Present |
| Load at 30% strain (N) | — | — | 83.5 | 49.1 | 64.0 |

### (Sensory Evaluation Method)

Six panelists ate various food compositions blindly, and evaluated “hardness,” “chewiness,” and “tastiness.” For each evaluation item, evaluation was performed in five stages according to the following evaluation criteria. The purpose of the present invention is to achieve a texture similar to that of meat; however, even if a texture similar to that of meat is attained, the food composition is not suitable if it has an off-flavor, an off-odor, or a sense of discomfort, and therefore, “tastiness” was also included as an evaluation criterion. The results obtained by sensory evaluation for each food composition of Examples 1 to 3 are shown in Table 4.

### (Evaluation Criteria)

Equivalent to natural meat materials such as livestock meat including mixed ground meat hamburger steak: 5

Similar to natural meat materials such as livestock meat including mixed ground meat hamburger steak, with a slight, non-significant sense of discomfort: 4

Similar to natural meat materials such as livestock meat including mixed ground meat hamburger steak, with a moderate sense of discomfort: 3

Different from natural meat materials such as livestock meat including mixed ground meat hamburger steak: 2

Completely different from natural meat materials such as livestock meat including mixed ground meat hamburger steak: 1

A total score of 6 or more across the three evaluation criteria was considered a pass, and a score of less than 6 was considered a fail.

TABLE 4

| | Comparative | Comparative | | | |
|---|---|---|---|---|---|
| | Example 1 | Example 2 | Example 1 | Example 2 | Example 3 |
| Hardness | — | — | 3.2 | 3.0 | 2.8 |
| Chewiness | — | — | 2.0 | 1.8 | 2.3 |
| Tastiness | — | — | 1.5 | 1.8 | 2.0 |
| Total score | — | — | 6.7 | 6.6 | 7.1 |
| Determination | — | — | Pass | Pass | Pass |

(Combination with Natural Meat Such as Livestock Meat)

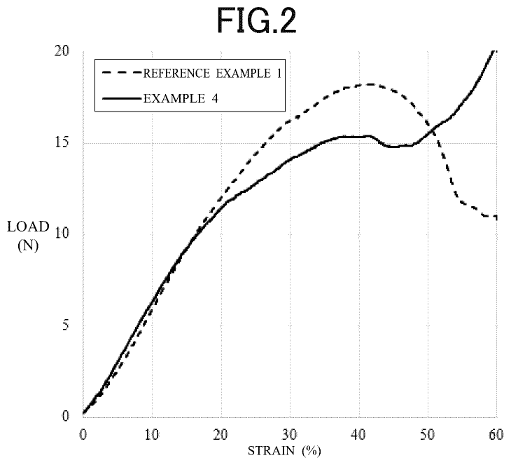

Each food composition of Example 4 and Reference Example 1 was prepared according to the formulation shown in Table 5. Pressed eringi was obtained by heating eringi ground to 3 mm or less with a food processor in a pot with a lid for about 10 minutes, and then reducing the mass from the original eringi to 40% with a pressing machine. As a transglutaminase preparation B, one containing 8% transglutaminase and 92% dextrin was used. The content of poorly soluble β-glucan, the moldability evaluation based on a load test, and the sensory evaluation were performed in the same manner as described above. The obtained results are shown in Tables 5 to 7 and FIG. 2.

TABLE 5

| | Structuring | Reference | |
|---|---|---|---|
| | material | Example 1 | Example 4 |
| Pressed eringi | — | — | 15.0 |
| Mixed ground meat | ◯ | 100.0 | 84.0 |
| Transglutaminase preparation B | ◯ (8%) | — | 1.0 |

| | | |
|---|---|---|
| Total | 100.0 | 100.0 |
| Mushroom solid content | — | 2.6 |
| Dietary fiber | — | 1.0 |
| Poorly soluble β-glucan | — | 0.32 |
| Structuring material | — | 84.1 |

(Unit of numerical values: mass %)

TABLE 6

| | Reference | |
|---|---|---|
| | Example 1 | Example 4 |
| Presence or absence of peak | Present | Present |
| Load at 30% strain (N) | 16.2 | 14.1 |

TABLE 7

| | Reference | |
|---|---|---|
| | Example 1 | Example 4 |
| Hardness | 4.7 | 4.5 |
| Chewiness | 4.8 | 4.3 |
| Tastiness | 4.8 | 4.5 |
| Total score | 14.3 | 13.3 |
| Determination | Pass | Pass |

### (Baked Product)

Food compositions were prepared according to the formulations shown in Table 8. The products of Example 5 and Comparative Example 3 before baking were molded and structured into a hamburger steak shape with a diameter of 7 cm and a thickness of 1.5 cm in the same manner as in Example 1. The resulting product was allowed to stand in a refrigerator for 20 hours, and then steamed for 15 minutes in a covered pot to which a small amount of water was added. Thereafter, salad oil was spread on a frying pan for baking, and heating was performed with an IH heater (IHK-TK62-B, Iris Ohyama Inc.) over medium heat until browning occurred, thereby obtaining Example 5 and Comparative Example 3.

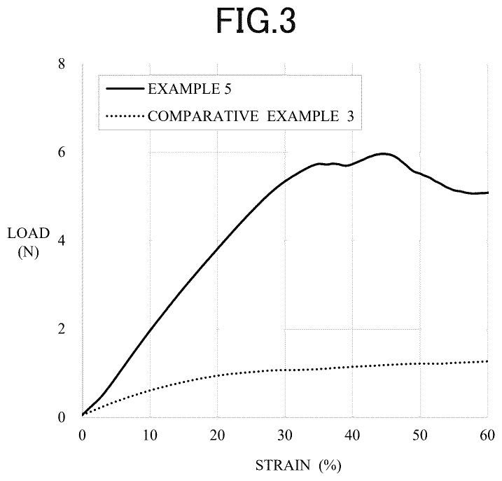

For each of the food compositions of Example 5 and Comparative Example 3, the content of poorly soluble β-glucan, the moldability evaluation based on a load test, and the sensory evaluation were performed in the same manner as described above. The obtained results are shown in Tables 8 to 10 and FIG. 3.

TABLE 8

| | Structuring | Example 5 | Comparative Example 3 |
|---|---|---|---|
| | material | before baking | before baking |
| Eringi | — | 50.0 | 50.0 |
| Transglutaminase preparation A | ◯ (93%) | 7.0 | — |
| Dextrin | — | — | 7.0 |
| Onion powder | — | 12.0 | 12.0 |
| Breadcrumbs | — | 10.0 | 10.0 |
| Onion | — | 20.0 | 20.0 |
| Salt and pepper | — | 1.0 | 1.0 |

| | | |
|---|---|---|
| Total | 100.0 | 100.0 |
| Mushroom solid content | 5.0 | 5.0 |
| Dietary fiber | 1.7 | 1.7 |
| Poorly soluble β-glucan | 0.99 | 0.99 |
| Structuring material | 6.5 | 0.0 |

| | | Heating and cooking |
|---|---|---|
| | Heating and cooking | Comparative |
| Processing | Example 5 | Example 3 |
| Mushroom solid content | 5.2 | 5.2 |
| Dietary fiber | 1.8 | 1.8 |
| Poorly soluble β-glucan | 1.04 | 1.04 |
| Structuring material | 6.8 | 0.0 |

(Unit of numerical values: mass %)

TABLE 9

| | | Comparative |
|---|---|---|
| | Example 5 | Example 3 |
| Presence or absence of peak | Present | Absent |
| Load at 30% strain (N) | 5.3 | 1.1 |

TABLE 10

| | | Comparative |
|---|---|---|
| | Example 5 | Example 3 |
| Hardness | 4.0 | 1.3 |
| Chewiness | 3.5 | 1.2 |
| Tastiness | 4.2 | 1.8 |
| Total score | 11.7 | 4.3 |
| Determination | Pass | Fail |

### (Derived Products Such as Meat Dumplings and Minced Meat Cutlets)

Food compositions were prepared according to the formulations shown in Table 11. Example 6 was structured in the same manner as in Example 1, and molded into a ball shape having a diameter of 2.5 cm. The product of Example 7 before processing was molded and structured in the same manner as in Example 1, the surface of the molded product was covered with a batter prepared by dissolving rice flour in water, and then coated with breadcrumbs on top. Thereafter, a food composition (processed product) was obtained by deep-frying in a pot with oil at 150° C. for 8 minutes.

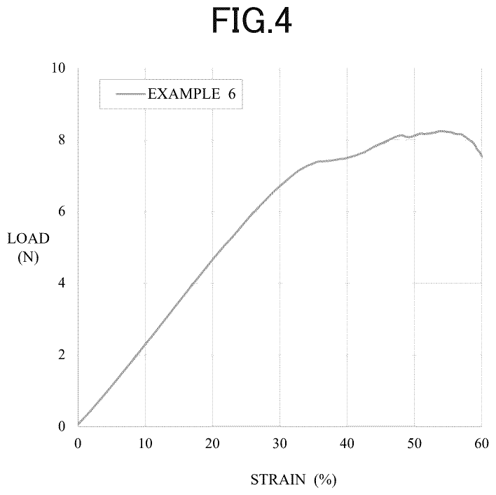

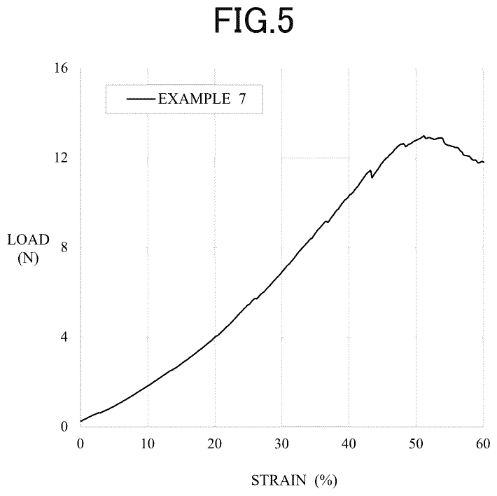

For each of the food compositions of Example 6 and Comparative Example 7, the content of poorly soluble β-glucan, the moldability evaluation based on a load test, and the sensory evaluation were performed in the same manner as described above. The obtained results are shown in Tables 11 to 13 and FIGS. 4 and 5.

The boiled lotus root used was “Boiled Whole Lotus Root” available from AEON Co., Ltd., the rice flour used was “Niigata no Komeko” available from Niigata Kenbei Co., Ltd., the breadcrumbs used were “FryStar Seven” available from FryStar Co., Ltd., and the salad oil used was “Nisshin Canola Oil” available from The Nisshin OilliO Group, Ltd.

TABLE 11

| | Structuring | | Example 7 |
|---|---|---|---|
| | material | Example 6 | before processing |
| Eringi | — | 50.0 | 39.0 |
| Transglutaminase preparation A | ◯ (93%) | 7.0 | 5.5 |
| Dextrin | — | — | — |
| Soy protein | ◯ | 10.0 | 3.9 |
| Onion powder | — | 7.0 | 5.5 |
| Breadcrumbs | — | 10.0 | 7.8 |
| Onion | — | 12.0 | 15.6 |
| Salt | — | 1.0 | 0.8 |
| Boiled lotus root | — | 3.0 | — |
| (Breading) | — | | 22.0 |
| Rice flour | — | | 8.3 |
| Water | — | | 8.3 |
| Breadcrumbs | — | | 5.4 |

| | | |
|---|---|---|
| Total | 100.0 | 100.0 |
| Mushroom solid content | 5.0 | 5.0 |
| Dietary fiber | 1.7 | 1.3 |
| Poorly soluble β-glucan | 0.99 | 0.77 |
| Structuring material | 16.5 | 9.0 |

| | Cooking with oil |
|---|---|
| Processing | Example 7 |
| Mushroom solid content | 5.5 |
| Dietary fiber | 1.5 |
| Poorly soluble β-glucan | 0.86 |
| Structuring material | 10.0 |

(Unit of numerical values: mass %)

TABLE 12

| | Example 6 | Example 7 |
|---|---|---|
| Presence or absence of peak | Present | Present |
| Load at 30% strain (N) | 6.7 | 6.9 |

TABLE 13

| | Example 6 | Example 7 |
|---|---|---|
| Hardness | 3.5 | 4.3 |
| Chewiness | 3.3 | 3.5 |
| Tastiness | 3.5 | 4.2 |
| Total score | 10.3 | 12.0 |
| Determination | Pass | Pass |

### (Dried and Rehydrated Product)

Structuring was performed in the same manner as in Example 1 according to the formulation shown in Table 14 to obtain a product before processing for Example 8, and then drying was performed in a dryer set at 60° C. for 20 hours to obtain a food composition of Example 8. Compared to the food composition before drying, Example 8 showed an 88% reduction in mass with a moisture content of 11.7%.

The food composition of Example 8 was rehydrated in a refrigerator set at 10° C. or lower for 2 hours, excess water was removed, and a food composition of Example 9 was obtained. Compared to the food composition of Example 8, the food composition of Example 9 showed a 38.6% increase in mass with a moisture content of 56.6%.

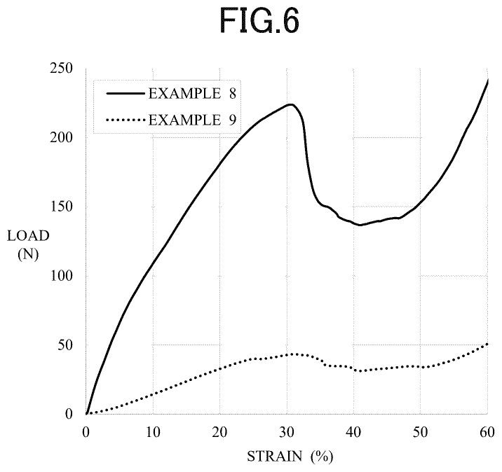

For each of the food compositions of Examples 8 and 9, the content of poorly soluble β-glucan, the moldability evaluation based on a load test, and the sensory evaluation were performed in the same manner as described above. The obtained results are shown in Tables 14 to 16 and FIG. 6.

TABLE 14

| | Structuring | Example 8 |
|---|---|---|
| | material | before processing |
| Pressed eringi | — | 95.0 |
| Transglutaminase preparation B | ◯ (8%) | 1.0 |
| Soy protein | ◯ | 4.0 |

| | |
|---|---|
| Total | 100 |
| Mushroom solid content | 16.2 |
| Dietary fiber | 6.5 |
| Poorly soluble β-glucan | 2.00 |
| Structuring material | 4.1 |

| | Dried | Dried and rehydrated |
|---|---|---|
| After processing | Example 8 | Example 9 |
| Mushroom solid content | 47.6 | 34.3 |
| Dietary fiber | 19.0 | 13.7 |
| Poorly soluble β-glucan | 5.90 | 4.25 |
| Structuring material | 12.1 | 8.7 |

(Unit of numerical values: mass %)

TABLE 15

| | Example 8 | Example 9 |
|---|---|---|
| Presence or absence of peak | Present | Present |
| Load at 30% strain (N) | 222.8 | 42.6 |

TABLE 16

| | Example 8 | Example 9 |
|---|---|---|
| Hardness | 2.5 | 3.7 |
| Chewiness | 2.7 | 3.3 |
| Tastiness | 2.7 | 3.4 |
| Total score | 7.9 | 10.7 |
| Determination | Pass | Pass |

### (Transglutaminase-Free, Sauce Immersion)

Food compositions were obtained in the same manner as in Example 1 according to the formulations in Table 17. As dextrin, “Sandec 185N” available from Sanwa Starch Co., Ltd. was used. In Example 11, the composition of Example 10 was immersed in “stewed hamburger steak sauce” available from Kagome Co., Ltd. and allowed to stand in a refrigerator set at 10° C. or lower for 24 hours. The moisture content of Example 10 was 59.3%, but the moisture content of Example 11 after immersion in the sauce increased to 71.8%.

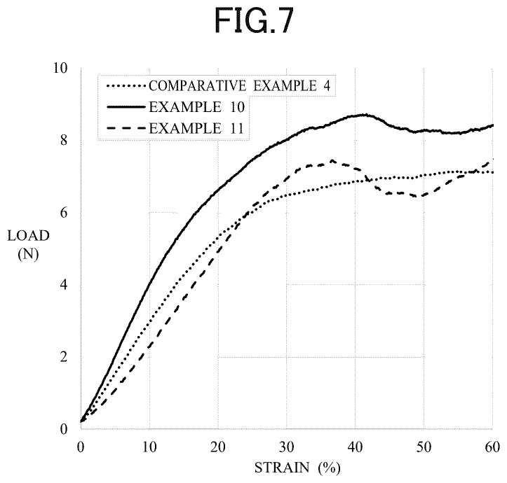

For each of the food compositions of Examples 10 and 11 and Comparative Example 4, the content of poorly soluble β-glucan, the moldability evaluation based on a load test, and the sensory evaluation were performed in the same manner as described above. The obtained results are shown in Tables 17 to 19 and FIG. 7.

TABLE 17

| | Structuring | Comparative | | Example 11 |
|---|---|---|---|---|
| | material | Example 4 | Example 10 | before processing |
| Pressed eringi | — | 95.0 | 95.0 | 95.0 |
| Transglutaminase preparation B | ◯ (8%) | 0.0 | 1.0 | 1.0 |
| Dextrin | — | 1.0 | 0.0 | 0.0 |
| Soy protein | ◯ | 4.0 | 4.0 | 4.0 |

| | | | |
|---|---|---|---|
| Total | 100.0 | 100.0 | 100.0 |
| Mushroom solid content | 16.2 | 16.2 | 16.2 |
| Dietary fiber | 6.5 | 6.5 | 6.5 |
| Poorly soluble β-glucan | 2.00 | 2.00 | 2.00 |
| Structuring material | 4.0 | 4.1 | 4.1 |

| | Sauce immersion |
|---|---|
| After processing | Example 11 |
| Mushroom solid content | 11.2 |
| Dietary fiber | 4.5 |
| Poorly soluble β-glucan | 1.39 |
| Structuring material | 2.8 |

(Unit of formulation: mass %)

TABLE 18

| | Comparative | | |
|---|---|---|---|
| | Example 4 | Example 10 | Example 11 |
| Presence or absence of peak | Absent | Present | Present |
| Load at 30% strain (N) | 6.5 | 8.0 | 6.9 |

TABLE 19

| | Comparative | | |
|---|---|---|---|
| | Example 4 | Example 10 | Example 11 |
| Hardness | 2.3 | 4.0 | 3.5 |
| Chewiness | 1.5 | 3.7 | 3.3 |
| Tastiness | 1.5 | 4.0 | 4.7 |
| Total score | 5.3 | 11.7 | 11.5 |
| Determination | Fail | Pass | Pass |

(Structuring with Alginate)

Each food composition was obtained by performing structuring according to the formulations shown in Table 20. A composition containing no sodium alginate preparation was regarded as Comparative Example 5, and compositions formulated with 1.6%, 3.2%, 4.8%, and 8.0% of a sodium alginate preparation were designated as Examples 12 to 15, respectively. The raw materials for Comparative Example 5 and Examples 12 to 15 were mixed well so as to be uniform, molded using a cylindrical mold having a diameter of 4 cm and a height of 3 cm, and allowed to stand in a refrigerator set at 10° C. or lower for 20 hours. Subsequently, steaming was performed for 15 minutes in a covered pot to which a small amount of water had been added, thereby obtaining each food composition. As a sodium alginate preparation, “Konbusan 429S” available from Kimica Corporation containing 63% of sodium alginate, 28% of calcium sulfate, and 9% of sodium pyrophosphate was used.

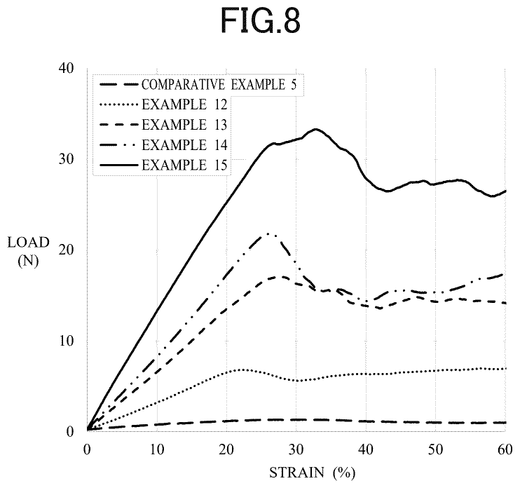

For each of the food compositions of Comparative Example 5 and Examples 12 to 15, the content of poorly soluble β-glucan, the moldability evaluation based on a load test, and the sensory evaluation were performed in the same manner as described above. The obtained results are shown in Tables 20 to 22 and FIG. 8.

TABLE 20

| | Structuring | Comparative | | | | |
|---|---|---|---|---|---|---|
| | material | Example 5 | Example 12 | Example 13 | Example 14 | Example 15 |
| Pressed eringi | | 100.0 | 98.4 | 96.8 | 95.2 | 92.0 |
| Sodium alginate preparation | ◯ (63%) | 0.0 | 1.6 | 3.2 | 4.8 | 8.0 |

| | | | | | |
|---|---|---|---|---|---|
| Total | 100.0 | 100.0 | 100.0 | 100.0 | 100.0 |
| Mushroom solid content | 17.0 | 16.7 | 16.5 | 16.2 | 15.6 |
| Dietary fiber | 6.8 | 6.7 | 6.6 | 6.5 | 6.3 |
| Poorly soluble β-glucan | 2.11 | 2.08 | 2.04 | 2.01 | 1.94 |
| Structuring material | 0.0 | 1.0 | 2.0 | 3.0 | 5.0 |

(Unit of numerical values: mass %)

TABLE 21

| | Comparative | | | | |
|---|---|---|---|---|---|
| | Example 5 | Example 12 | Example 13 | Example 14 | Example 15 |
| Presence or absence of peak | Absent | Present | Present | Present | Present |
| Load at 30% strain (N) | 1.3 | 5.6 | 16.3 | 18.6 | 32.1 |

TABLE 22

| | Comparative | | | | |
|---|---|---|---|---|---|
| | Example 5 | Example 12 | Example 13 | Example 14 | Example 15 |
| Hardness | 1.3 | 2.5 | 3.3 | 3.2 | 2.5 |
| Chewiness | 1.5 | 2.3 | 2.7 | 2.8 | 3.0 |
| Tastiness | 1.5 | 1.5 | 1.8 | 2.2 | 1.5 |
| Total score | 4.3 | 6.3 | 7.8 | 8.2 | 7.0 |
| Determination | Fail | Pass | Pass | Pass | Pass |

(Structuring with Mannans)

Each food composition of Example 16 and Comparative Examples 6 to 8 was prepared according to the formulation shown in Table 23. Example 16 and Comparative Examples 6 to 8 were prepared by mixing konjac powder dispersed in oil with eringi mushrooms, allowing the mixture to stand for a certain period of time for structuring, thereby forming a mannan base, followed by mixing with other materials and stirring with a multi-blender, and then molding in the same manner as in Example 1. Ultramannan G5 manufactured by Ina Food Industry Co., Ltd. was used as konjac powder.

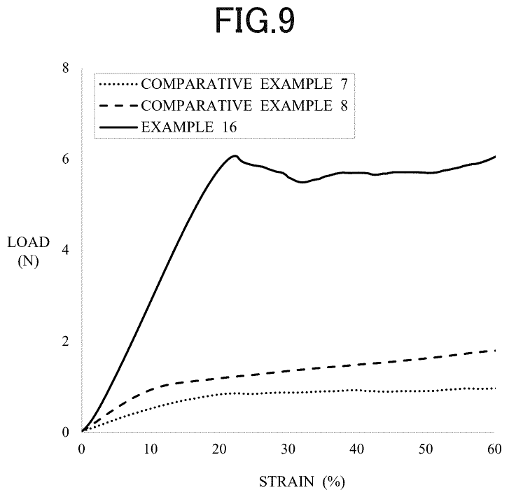

For each of the food compositions of Example 16 and Comparative Examples 6 to 8, the contents of dietary fiber and poorly soluble β-glucan, the physical property evaluation based on a load test, and the sensory evaluation were performed in the same manner as described above. The obtained results are shown in Tables 23 to 25 and FIG. 9.

TABLE 23

| | | Structuring | Comparative | Comparative | Comparative | |
|---|---|---|---|---|---|---|
| | | material | Example 6 | Example 7 | Example 8 | Example 16 |
| | Standing time (hours) | | 1.0 | 1.0 | 0.0 | 1.0 |
| Mannan | Eringi | — | 39.0 | 39.0 | 39.0 | 39.0 |
| base | Oil | — | 6.0 | 5.0 | 5.0 | 5.0 |
| | Konjac powder | ◯ | 0.0 | 1.0 | 1.0 | 1.0 |
| | Eringi | — | 35.0 | 35.0 | 15.0 | 15.0 |
| | Soy protein | ◯ | 0.0 | 0.0 | 20.0 | 20.0 |
| | Water | — | 20.0 | 20.0 | 20.0 | 20.0 |

| | | | | |
|---|---|---|---|---|
| Total | 100.0 | 100.0 | 100.0 | 100.0 |
| Mushroom solid content | 7.4 | 7.4 | 5.4 | 5.4 |
| Dietary fiber | 2.5 | 2.5 | 1.8 | 1.8 |
| Poorly soluble β-glucan | 1.5 | 1.5 | 1.1 | 1.1 |
| Structuring material | 0.0 | 1.0 | 21.0 | 21.0 |

(Unit of numerical values: mass %)

TABLE 24

| | Comparative | Comparative | Comparative | |
|---|---|---|---|---|
| | Example 6 | Example 7 | Example 8 | Example 16 |
| Presence or absence of peak | Unmoldable | Absent | Absent | Present |
| Load at 30% strain (N) | — | 0.86 | 1.34 | 5.60 |

TABLE 25

| | Comparative | Comparative | Comparative | |
|---|---|---|---|---|
| | Example 6 | Example 7 | Example 8 | Example 16 |
| Hardness | — | 1.2 | 1.7 | 2.8 |
| Chewiness | — | 1.2 | 1.5 | 2.7 |
| Tastiness | — | 1.0 | 1.3 | 2.2 |
| Total score | — | 3.3 | 4.5 | 7.7 |
| Determination | — | Fail | Fail | Pass |

### (Shape of Mushrooms)

In order to examine the effect of the shape of mushrooms, food compositions were prepared using eringi processed into different shapes according to the formulations shown in Table 26. That is, one obtained by grinding with a food processor and using the eringi having a particle size of 3 mm or less was used as Example 17, and one obtained by using eringi adjusted to a particle size of 3 to 6 mm was used as Example 18 in the same manner. In addition, one prepared using eringi that had been torn by hand to a thickness of about 7 mm and a length of about 5 cm, and adjusted into a strip shape was used as Example 19. These eringi samples having different shapes were ground, steamed for 10 minutes, and then pressed so as to have a compression ratio of 40%, and used for the test. The structuring was performed in the same manner as in Example 1. The sugar used was “White Sugar” from Nissin Sugar Co., Ltd., and the salt used was “Hakata no Shio Ara-shio” from Hakata Salt Co., Ltd. The white pepper used was “White Pepper” available from Gaban Co., Ltd.

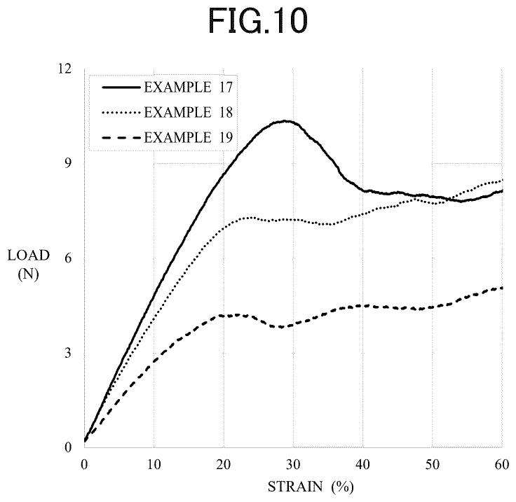

For each of the food compositions of Examples 17 to 19, the content of poorly soluble β-glucan, the moldability evaluation based on a load test, and the sensory evaluation were performed in the same manner as described above. The obtained results are shown in Tables 26 to 28 and FIG. 10.

TABLE 26

| | Structuring | Example 17 | Example 18 | Example 19 |
|---|---|---|---|---|
| Shape | material | 3 mm or less | 3 to 6 mm | Strip shape |
| Pressed eringi | — | 64.8 | 64.8 | 64.8 |
| Transglutaminase preparation A | ◯ (93%) | 6.5 | 6.5 | 6.5 |
| Breadcrumbs | — | 13.5 | 13.5 | 13.5 |
| Sugar | — | 1.8 | 1.8 | 1.8 |
| Salt | — | 1.4 | 1.4 | 1.4 |
| White pepper | — | 0.1 | 0.1 | 0.1 |
| Water | — | 11.9 | 11.9 | 11.9 |

| | | | |
|---|---|---|---|
| Total | 100.0 | 100.0 | 100.0 |
| Mushroom solid content | 11.0 | 11.0 | 11.0 |
| Dietary fiber | 4.4 | 4.4 | 4.4 |
| Poorly soluble β-glucan | 1.37 | 1.37 | 1.37 |
| Structuring material | 6.0 | 6.0 | 6.0 |

(Unit of formulation: mass %)

TABLE 27

| | Example 17 | Example 18 | Example 19 |
|---|---|---|---|
| Presence or absence of peak | Present | Present | Present |
| Load at 30% strain (N) | 10.3 | 7.2 | 3.9 |

TABLE 28

| | Example 17 | Example 18 | Example 19 |
|---|---|---|---|
| Hardness | 3.5 | 3.0 | 2.8 |
| Chewiness | 2.5 | 3.0 | 2.3 |
| Tastiness | 4.0 | 3.8 | 2.5 |
| Total score | 10.0 | 9.8 | 7.6 |
| Determination | Pass | Pass | Pass |

### (Amount of Non-Mushroom-Derived Protein Material)

Food compositions of Comparative Example 9 and Examples 20 to 23 were obtained in the same manner as in Example 1 according to the formulations shown in Table 29. The pea protein used was “Pea Protein Derived from Peas” available from Usuki Pharmaceutical Co., Ltd.

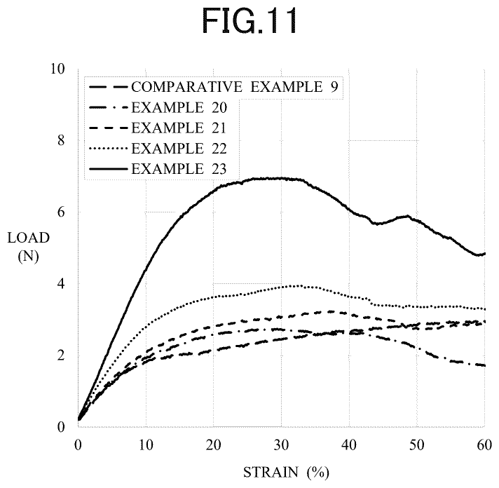

For each of the food compositions of Comparative Example 9 and Examples 20 to 23, the content of poorly soluble β-glucan, the moldability evaluation based on a load test, and the sensory evaluation were performed in the same manner as described above. The obtained results are shown in Tables 29 to 31 and FIG. 11.

TABLE 29

| | Structuring | Comparative | | | | |
|---|---|---|---|---|---|---|
| | material | Example 9 | Example 20 | Example 21 | Example 22 | Example 23 |
| Pressed eringi | — | 80.0 | 80.0 | 80.0 | 80.0 | 80.0 |
| Transglutaminase preparation B | ◯ (8%) | 1.0 | 1.0 | 1.0 | 1.0 | 1.0 |
| Dextrin | — | 19.0 | 17.0 | 15.0 | 13.0 | 9.0 |
| Pea protein | ◯ | 0.0 | 2.0 | 4.0 | 6.0 | 10.0 |

| | | | | | |
|---|---|---|---|---|---|
| Total | 100.0 | 100.0 | 100.0 | 100.0 | 100.0 |
| Mushroom solid content | 13.6 | 13.6 | 13.6 | 13.6 | 13.6 |
| Dietary fiber | 5.4 | 5.4 | 5.4 | 5.4 | 5.4 |
| Poorly soluble β-glucan | 1.69 | 1.69 | 1.69 | 1.69 | 1.69 |
| Structuring material | 0.1 | 2.1 | 4.1 | 6.1 | 10.1 |

(Unit of numerical values: mass %)

TABLE 30

| | Comparative | | | | |
|---|---|---|---|---|---|
| | Example 9 | Example 20 | Example 21 | Example 22 | Example 23 |
| Presence or absence of peak | Absent | Present | Present | Present | Present |
| Load at 30% strain (N) | 2.4 | 2.7 | 3.1 | 3.9 | 7.0 |

TABLE 31

| | Comparative | | | | |
|---|---|---|---|---|---|
| | Example 9 | Example 20 | Example 21 | Example 22 | Example 23 |
| Hardness | 1.7 | 2.3 | 2.7 | 3.2 | 3.5 |
| Chewiness | 1.3 | 2.0 | 2.7 | 2.8 | 3.3 |
| Tastiness | 1.7 | 2.2 | 2.5 | 2.8 | 2.7 |
| Total score | 4.7 | 6.5 | 7.9 | 8.8 | 9.5 |
| Determination | Fail | Pass | Pass | Pass | Pass |

### (Mushroom Species and Combinations)

Food compositions were prepared using various mushrooms or a plurality of mushrooms according to the formulations shown in Table 32. Examples 24 to 29 and 31 were molded and structured in the same manner as in Example 1, and Examples 30 and 32 were molded and structured in the same manner as in Example 1, except that ones processed in advance in an autoclave at 121° C. for 20 minutes were used.

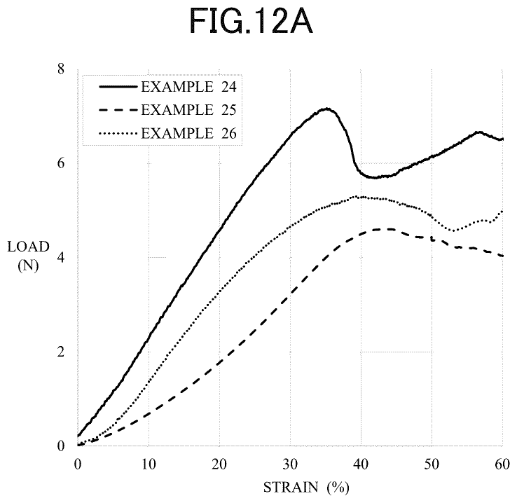

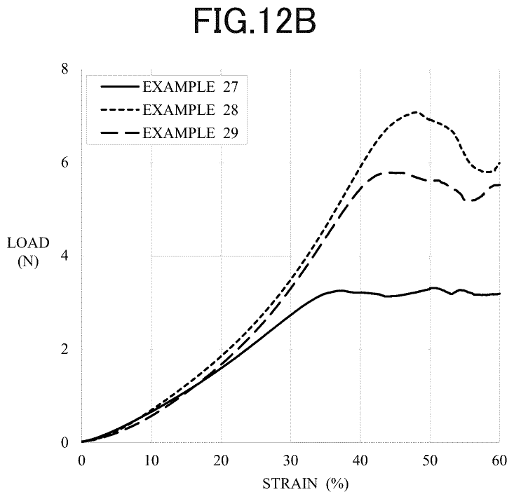

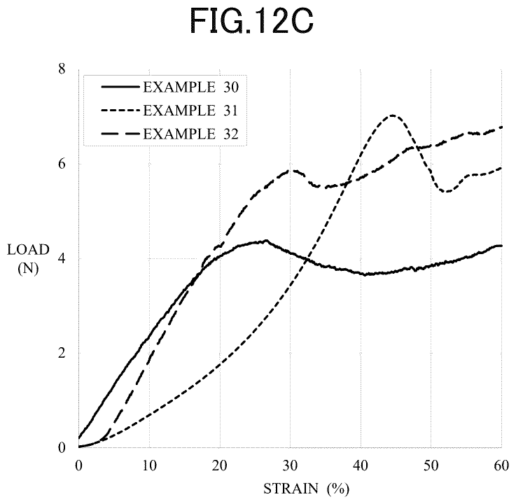

For each of the food compositions of Examples 24 to 32, the content of poorly soluble β-glucan, the moldability evaluation based on a load test, and the sensory evaluation were performed in the same manner as described above. The obtained results are shown in Tables 32 to 34 and FIGS. 12A to 12C.

TABLE 32

| | Structuring | Example | Example | Example | Example | Example | Example | Example | Example | Example |
|---|---|---|---|---|---|---|---|---|---|---|
| | material | 24 | 25 | 26 | 27 | 28 | 29 | 30 | 31 | 32 |
| Eringi | — | 50.0 | — | — | — | — | — | — | — | 35.0 |
| Enokitake | — | — | 50.0 | — | — | — | — | — | — | — |
| Wood ear mushroom | — | — | — | 50.0 | — | — | — | — | — | — |
| Shiitake | — | — | — | — | 50.0 | — | — | — | — | — |
| Oyster mushroom | — | — | — | — | — | 50.0 | — | — | — | — |
| Buna-shimeji | — | — | — | — | — | — | 50.0 | — | — | — |
| Maitake | — | — | — | — | — | — | — | 50.0 | — | 15.0 |
| Button mushroom | — | — | — | — | — | — | — | — | 50.0 | — |
| Transglutaminase | ◯ (93%) | 7.0 | 7.0 | 7.0 | 7.0 | 7.0 | 7.0 | 7.0 | 7.0 | 7.0 |
| preparation A | | | | | | | | | | |
| Soy protein | ◯ | 5.0 | 5.0 | 5.0 | 5.0 | 5.0 | 5.0 | 5.0 | 5.0 | 5.0 |
| Onion powder | — | 7.0 | 7.0 | 7.0 | 7.0 | 7.0 | 7.0 | 7.0 | 7.0 | 7.0 |
| Breadcrumbs | — | 10.0 | 10.0 | 10.0 | 10.0 | 10.0 | 10.0 | 10.0 | 10.0 | 10.0 |
| Water | — | 20.0 | 20.0 | 20.0 | 20.0 | 20.0 | 20.0 | 20.0 | 20.0 | 20.0 |
| Salt and pepper | — | 1.0 | 1.0 | 1.0 | 1.0 | 1.0 | 1.0 | 1.0 | 1.0 | 1.0 |

| | | | | | | | | | |
|---|---|---|---|---|---|---|---|---|---|
| Total | 100.0 | 100.0 | 100.0 | 100.0 | 100.0 | 100.0 | 100.0 | 100.0 | 100.0 |
| Mushroom solid content | 5.0 | 5.7 | 3.2 | 5.2 | 5.3 | 4.5 | 3.7 | 3.1 | 4.6 |
| Dietary fiber | 1.7 | 2.0 | 2.4 | 2.5 | 1.3 | 1.5 | 1.8 | 2.0 | 1.7 |
| Poorly soluble β-glucan | 0.99 | 0.30 | 0.07 | 0.33 | 0.60 | 0.52 | 0.69 | 0.07 | 0.90 |
| Structuring material | 11.5 | 11.5 | 11.5 | 11.5 | 11.5 | 11.5 | 11.5 | 11.5 | 11.5 |

(Unit of numerical values: mass %)

TABLE 33

| | Example | Example | Example | Example | Example | Example | Example | Example | Example |
|---|---|---|---|---|---|---|---|---|---|
| | 24 | 25 | 26 | 27 | 28 | 29 | 30 | 31 | 32 |
| Presence or absence of peak | Present | Present | Present | Present | Present | Present | Present | Present | Present |
| Load at 30% strain (N) | 6.6 | 3.2 | 4.7 | 2.7 | 3.5 | 3.3 | 4.1 | 3.4 | 5.7 |

TABLE 34

| | Example | Example | Example | Example | Example | Example | Example | Example | Example |
|---|---|---|---|---|---|---|---|---|---|
| | 24 | 25 | 26 | 27 | 28 | 29 | 30 | 31 | 32 |
| Hardness | 4.3 | 3.3 | 3.5 | 2.7 | 3.8 | 3.3 | 2.5 | 3.7 | 3.8 |
| Chewiness | 3.5 | 2.0 | 2.3 | 2.5 | 2.3 | 2.5 | 2.2 | 2.0 | 3.3 |
| Tastiness | 4.2 | 3.7 | 2.5 | 1.7 | 1.7 | 2.3 | 2.3 | 2.3 | 3.8 |
| Total score | 12.0 | 9.0 | 8.3 | 6.9 | 7.8 | 8.1 | 7.0 | 8.0 | 10.9 |
| Determination | Pass | Pass | Pass | Pass | Pass | Pass | Pass | Pass | Pass |

### (Influence of Protease Inherent in Mushrooms)

Eringi ground to 3 mm or less using a food processor and treated in an autoclave at 121° C. for 20 minutes was designated as eringi (autoclaved). Molding and structuring were performed in the same manner as in Example 1 according to the formulations shown in Table 35.

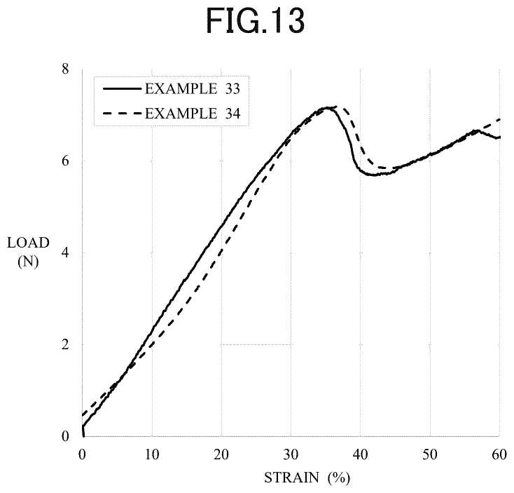

For each of the food compositions of Examples 33 to 34, the content of poorly soluble β-glucan, the moldability evaluation based on a load test, and the sensory evaluation were performed in the same manner as described above. The obtained results are shown in Tables 35 to 37 and FIG. 13.

TABLE 35

| | Structuring | | |
|---|---|---|---|
| | material | Example 33 | Example 34 |
| Eringi | — | 50.0 | — |
| Eringi (autoclaved) | — | — | 50.0 |
| Transglutaminase | ◯ (93%) | 7.0 | 7.0 |
| preparation A | | | |
| Soy protein | ◯ | 5.0 | 5.0 |
| Onion powder | — | 7.0 | 7.0 |
| Breadcrumbs | — | 10.0 | 10.0 |
| Water | — | 20.0 | 20.0 |
| Salt and pepper | — | 1.0 | 1.0 |

| | | |
|---|---|---|
| Total | 100.0 | 100.0 |
| Mushroom solid content | 5.0 | 5.0 |
| Dietary fiber | 1.7 | 1.7 |
| Poorly soluble β-glucan | 0.99 | 0.99 |
| Structuring material | 11.5 | 11.5 |

(Unit of numerical values: mass %)

TABLE 36

| | Example 33 | Example 34 |
|---|---|---|
| Presence or absence of peak | Present | Present |
| Load at 30% strain (N) | 6.6 | 6.5 |

TABLE 37

| | Example 33 | Example 34 |
|---|---|---|
| Hardness | 4.3 | 4.3 |
| Chewiness | 3.5 | 3.8 |
| Tastiness | 4.2 | 4.2 |
| Total score | 12.0 | 12.3 |
| Determination | Pass | Pass |

### (Addition of Alginate)

Each food composition of Example 35 and Comparative Examples 10 and 11 was prepared in the same manner as in Example 12 according to the formulations shown in Table 38.

The sodium alginate preparation used was “Konbusan 429S” available from Kimica Corporation. The methyl cellulose mixture used was prepared by mixing 36 weight % of “Veggie-Meat Helper,” a methyl cellulose formulation available from The Torigoe Co., Ltd., with 36 weight % of “Nisshin Canola Oil” available from The Nisshin OilliO Group, Ltd., and 150 weight % of cold water, in those proportions, using a multi-blender (Kai Select 100, available from KAI Corporation) for 3 minutes. The pectin used was “Apple Pectin HM” available from Unitec Foods Co., Ltd.

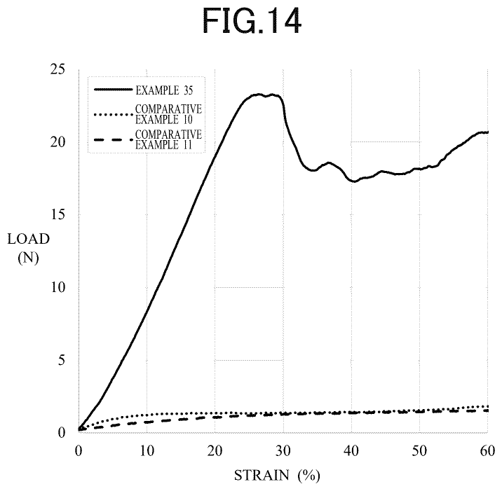

For each of the food compositions of Example 35 and Comparative Examples 10 and 11, the content of poorly soluble β-glucan, the sensory evaluation, and the moldability evaluation based on a load test were performed in the same manner as described above. The obtained results are shown in Tables 38 to 40 and FIG. 14.

TABLE 38

| | Structuring | | Comparative | Comparative |
|---|---|---|---|---|
| | material | Example 35 | Example 10 | Example 11 |
| Pressed eringi | — | 80.0 | 80.0 | 80.0 |
| Sodium alginate preparation | ◯ (63%) | 3.0 | — | — |
| Methyl cellulose mixture | — | — | 10.0 | — |
| Pectin | — | — | — | 10.0 |
| Dextrin | — | 17.0 | 10.0 | 10.0 |

| | | | |
|---|---|---|---|
| Total | 100.0 | 100.0 | 100.0 |
| Mushroom solid content | 13.6 | 13.6 | 13.6 |
| Dietary fiber | 5.4 | 5.4 | 5.4 |
| Poorly soluble β-glucan | 1.69 | 1.69 | 1.69 |
| Structuring material | 1.9 | 0.0 | 0.0 |

(Unit of numerical values: mass %)

TABLE 39

| | | Comparative | Comparative |
|---|---|---|---|
| | Example 35 | Example 10 | Example 11 |
| Presence or absence of peak | Present | Absent | Absent |
| Load at 30% strain (N) | 22.6 | 1.4 | 1.3 |

TABLE 40

| | | Comparative | Comparative |
|---|---|---|---|
| | Example 35 | Example 10 | Example 11 |
| Hardness | 3.5 | 1.8 | 1.5 |
| Chewiness | 2.5 | 1.7 | 2.2 |
| Tastiness | 1.7 | 2.2 | 1.3 |
| Total score | 7.7 | 5.7 | 5.0 |
| Determination | Pass | Fail | Fail |

### (Combined Use of Polysaccharides and Enzyme)

In order to examine the effect of combined use of structuring materials, food compositions of Examples 36 to 39 were prepared in the same manner as in Example 1 according to the formulations shown in Table 41. The curdlan used was “Curdlan” available from FUJIFILM Wako Pure Chemical Corporation. The konjac powder mixture used was prepared by formulating 97 mass % of konjac powder, “Shirayuki Tokujo” available from Kitamura Liquor store Corporation., with 3% of “Calcium Hydroxide,” a coagulant available from Kitamura Liquor store Corporation.

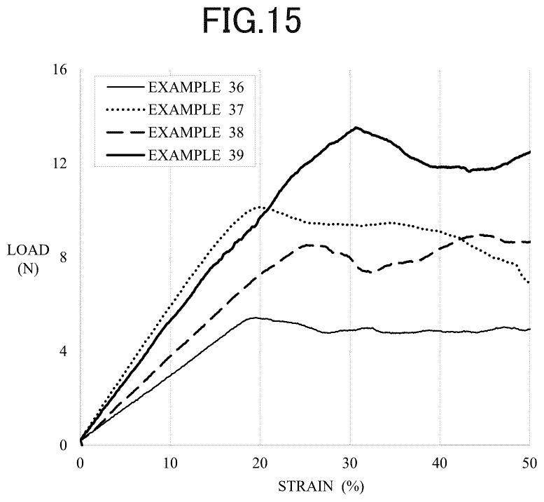

For each of the food compositions of Examples 36 to 39, the content of poorly soluble β-glucan, the moldability evaluation based on a load test, and the sensory evaluation were performed in the same manner as described above. The obtained results are shown in Tables 41 to 43 and FIG. 15.

TABLE 41

| | Structuring | | Example | Example | Example | Example |
|---|---|---|---|---|---|---|
| | material | Polysaccharides | 36 | 37 | 38 | 39 |
| Pressed eringi | — | — | 80.0 | 80.0 | 80.0 | 80.0 |
| Transglutaminase preparation A | ◯ (93%) | — | 7.0 | 7.0 | 7.0 | 7.0 |
| Dextrin | — | — | 13.0 | 10.0 | 10.0 | 10.0 |
| Polysaccharides | ◯ (63%) | Sodium alginate preparation | — | 3.0 | — | — |
| used in combination | — | Curdlan | — | — | 3.0 | — |
| | ◯ | Konjac powder | — | — | — | 3.0 |

| | | | | |
|---|---|---|---|---|
| Total | 100.0 | 100.0 | 100.0 | 100.0 |
| Mushroom solid content | 13.6 | 13.6 | 13.6 | 13.6 |
| Dietary fiber | 5.4 | 5.4 | 5.4 | 5.4 |
| Poorly soluble β-glucan | 1.69 | 1.69 | 1.69 | 1.69 |
| Structuring material | 6.5 | 8.4 | 6.5 | 9.5 |

(Unit of numerical values: mass %)

TABLE 42

| | Example | Example | Example | Example |
|---|---|---|---|---|
| | 36 | 37 | 38 | 39 |
| Presence or | Present | Present | Present | Present |
| absence of peak | | | | |
| Load at 30% strain (N) | 4.9 | 9.4 | 8.0 | 13.3 |

TABLE 43

| | Example | Example | Example | Example |
|---|---|---|---|---|
| | 36 | 37 | 38 | 39 |
| Hardness | 2.2 | 3.7 | 3.2 | 4.2 |
| Chewiness | 3.3 | 3.5 | 2.5 | 3.2 |
| Tastiness | 3.0 | 2.8 | 3.5 | 3.5 |
| Total score | 8.5 | 10.0 | 9.2 | 10.9 |
| Determination | Pass | Pass | Pass | Pass |

### (Properties of Mushroom Itself)

In the formulations shown in Table 44, eringi cut into pieces 2.0 cm in length, 2.0 cm in width, and approximately 1.0 cm in thickness was designated as Comparative Example 12. A dried eringi product obtained by drying Comparative Example 12 at 70° C. for 2 hours and 30 minutes to achieve a moisture content of 59.0% was designated as Comparative Example 13. The dried eringi material used was prepared by sealing in a plastic bag to eliminate moisture unevenness and storing in a refrigerator set at 10° C. or lower for 16 hours.

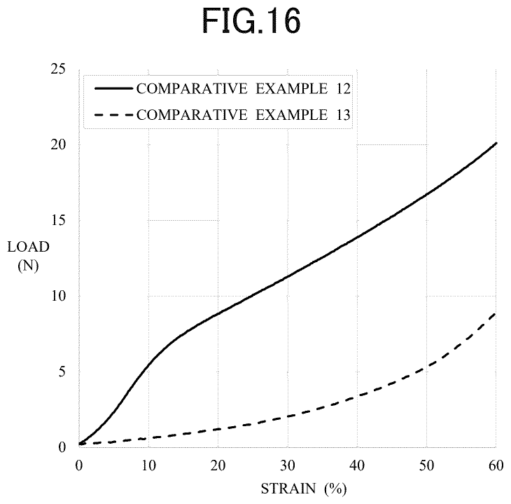

For each of the food compositions of Comparative Examples 12 and 13, the content of poorly soluble β-glucan, the sensory evaluation, and the moldability evaluation based on a load test were performed in the same manner as described above. The obtained results are shown in Tables 44 to 46 and FIG. 16.

TABLE 44

| | Comparative | Comparative |
|---|---|---|
| | Example 12 | Example 13 |
| Eringi | 100.0 | 100.0 |
| Moisture content | 90.0 | 59.0 |
| Mushroom solid content | 10.0 | 41.0 |
| Dietary fiber | 3.4 | 15.6 |
| Poorly soluble β-glucan | 1.98 | 8.12 |
| Structuring material | 0.0 | 0.0 |

(Unit of numerical values: mass %)

TABLE 45

| | Comparative | Comparative |
|---|---|---|
| | Example 12 | Example 13 |
| Presence or absence of peak | Absent | Absent |
| Load at 30% strain (N) | 11.3 | 2.0 |

TABLE 46

| | Comparative | Comparative |
|---|---|---|
| | Example 12 | Example 13 |
| Hardness | 2.7 | 1.3 |
| Chewiness Tastiness | 1.3 | 1.5 |
| | 1.0 | 1.2 |
| Total score | 5.0 | 4.0 |
| Determination | Fail | Fail |

### (Comparison Between Fruiting Body and Mycelium)

Using an eringi fruiting body and an eringi mycelium as materials, molded and structured products in the same manner as in Example 1 according to the formulations shown in Table 48 were designated as Examples 40 and 41, respectively. The eringi mycelium was obtained by the following method.

Eringi grown on PDA agar medium at 26° C. for one week was aseptically inoculated into the PDA liquid medium shown in Table 47, and subjected to rotary shaking culture at 26° C. for two weeks at 80 rpm. Shaking was performed in a 500 mL Erlenmeyer flask with baffles. The above cultured product was heated to 90° C. and then centrifuged at 7000 rpm for 25 minutes, and the supernatant was removed to obtain the eringi mycelium.

The soy protein used was “Soy Protein” available from Nippon Garlic Corporation, the onion powder used was “Onion Powder” available from Youki Food Co., Ltd., and the salt and pepper used was “Ajitsuke Shio Kosho” available from S&B Foods Inc.

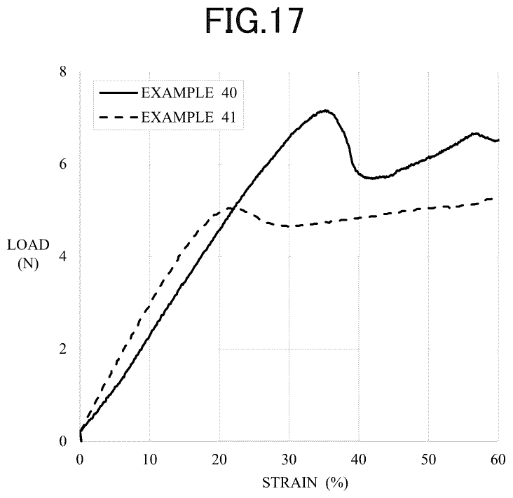

For each of the food compositions of Examples 40 and 41, the content of poorly soluble β-glucan, the moldability evaluation based on a load test, and the sensory evaluation were performed in the same manner as described above. The obtained results are shown in Tables 48 to 50 and FIG. 17.

TABLE 47

| Details | Content |
|---|---|
| Glucose | 2.0 |
| Potassium dihydrogen phosphate | 0.05 |
| Magnesium sulfate heptahydrate | 0.05 |
| High polypeptone S | 0.2 |
| Yeast extract | 0.2 |
| Distilled water | 97.5 |
| Total | 100 |

(Unit of numerical values: mass %)

TABLE 48

| | Structuring | Example | Example |
|---|---|---|---|
| | material | 40 | 41 |
| Eringi fruiting body | — | 50.0 | — |
| Eringi mycelium | — | — | 50.0 |
| Transglutaminase preparation A | ◯ (93%) | 7.0 | 7.0 |
| Soy protein | ◯ | 5.0 | 5.0 |
| Onion powder | — | 7.0 | 7.0 |
| Breadcrumbs | — | 10.0 | 10.0 |
| Water | — | 20.0 | 20.0 |
| Salt and pepper | — | 1.0 | 1.0 |

| | | |
|---|---|---|
| Total | 100.0 | 100.0 |
| Mushroom solid content | 5.0 | 5.0 |
| Dietary fiber | 1.7 | 0.9 |
| Poorly soluble β-glucan | 0.99 | 0.56 |
| Structuring material | 11.5 | 11.5 |

(Unit of numerical values: mass %)

TABLE 49

| | Example | Example |
|---|---|---|
| | 40 | 41 |
| Presence or absence of peak | Present | Present |
| Load at 30% strain (N) | 6.6 | 4.7 |

TABLE 50

| | Example | Example |
|---|---|---|
| | 40 | 41 |
| Hardness | 4.3 | 2.3 |
| Chewiness | 3.8 | 2.5 |
| Tastiness | 4.2 | 3.7 |
| Total score | 12.3 | 8.5 |
| Determination | Pass | Pass |

### (Influence of Pre-Squeezing and Pre-Drying)

Food compositions were produced with pre-squeezed and pre-dried mushrooms in order to incorporate a large amount of fungi-derived poorly soluble β-glucan. The formulations were based on Table 51, and for Example 42, eringi was ground to 3 mm or less using a food processor and then dehydrated to 40% of its original weight using a pressing machine, and the resulting product was used as “pressed eringi.” For Example 43, eringi was ground to 3 mm or less using a food processor, then heated in a frying pan, and similarly dried and dehydrated to 40% of its original weight, and the resulting product was used as “heated eringi.” The other steps were performed in the same manner as in Example 1 for molding and structuring, and the resulting products were designated as Examples 42 and 43, respectively.

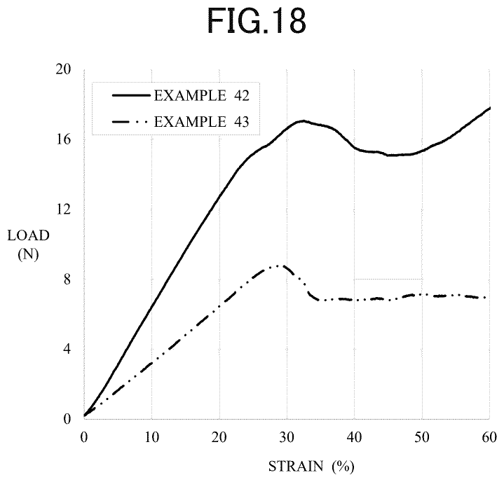

For each of the food compositions of Examples 42 and 43, the content of poorly soluble β-glucan, the moldability evaluation based on a load test, and the sensory evaluation were performed in the same manner as described above. The obtained results are shown in Tables 51 to 53 and FIG. 18.

TABLE 51

| | Structuring | | |
|---|---|---|---|
| | material | Example 42 | Example 43 |
| Heated eringi | — | — | 85.0 |
| Pressed eringi | — | 85.0 | — |
| Transglutaminase | ◯ (93%) | 7.0 | 7.0 |
| preparation A | | | |
| Soy protein | ◯ | 2.5 | 2.5 |
| Salad oil | — | 5.0 | 5.0 |
| Salt and pepper | — | 0.5 | 0.5 |

| | | |
|---|---|---|
| Total | 100.0 | 100.0 |
| Mushroom solid content | 14.5 | 14.5 |
| Dietary fiber | 5.8 | 5.8 |
| Thinh | | |
| Poorly soluble β-glucan | 1.79 | 1.79 |
| Structuring material | 9.0 | 9.0 |

(Unit of numerical values: mass %)

TABLE 52

| | Example 42 | Example 43 |
|---|---|---|
| Presence or absence of peak | Present | Present |
| Load at 30% strain (N) | 16.6 | 8.8 |

TABLE 53

| | Example 42 | Example 43 |
|---|---|---|
| Hardness | 4.0 | 3.7 |
| Chewiness | 3.3 | 3.7 |
| Tastiness | 3.3 | 3.2 |
| Total score | 10.6 | 10.6 |
| Determination | Pass | Pass |

### (Non-Preheated and Non-Stir-Fried Products)

In the formulation shown in Table 54, to confirm whether heating or stir-frying is necessary prior to structuring, a composition was produced without heating or stir-frying. Example 44 was molded and structured in the same manner as in Example 1, except that eringi was ground to 3 mm or less, then pressed to 40% of its original weight using a pressing machine without heating or stir-frying, and the resulting product was used as “pressed eringi.”

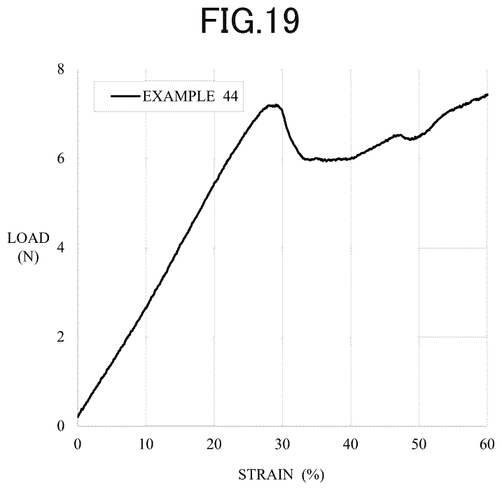

For each of the food compositions of Example 44, the content of poorly soluble β-glucan, the moldability evaluation based on a load test, and the sensory evaluation were performed in the same manner as described above. The obtained results are shown in Tables 54 to 56 and FIG. 19.

TABLE 54

| | Structuring | |
|---|---|---|
| | material | Example 44 |
| Pressed eringi | — | 68.2 |
| Transglutaminase preparation A | ◯ (93%) | 6.8 |
| Breadcrumbs | — | 10.0 |
| Sugar | — | 1.4 |
| Salt | — | 1.0 |
| White pepper | — | 0.1 |
| Water | — | 12.5 |

| | |
|---|---|
| Total | 100.0 |
| Mushroom solid content | 11.6 |
| Dietary fiber | 4.6 |
| Poorly soluble β-glucan | 1.44 |
| Structuring material | 6.3 |

(Unit of formulation: mass %)

TABLE 55

| | Example 44 |
|---|---|
| Presence or absence of peak | Present |
| Load at 30% strain (N) | 7.1 |

TABLE 56

| | Example 44 |
|---|---|
| Hardness | 3.7 |
| Chewiness | 2.8 |
| Tastiness | 3.3 |
| Total score | 9.8 |
| Determination | Pass |

(Comparison with Common Binding)

According to the formulations shown in Table 57, Example 45 was procuced in the same manner as in Example 1, and Comparative Example 14 was produced by the following method.

That is, in Comparative Example 14, eringi ground to 3 mm or less using a food processor was stir-fried for 3 minutes in a frying pan with 5.3 mass % of oil over medium heat, after which 15.0 mass % of onion diced into 1 to 2 mm cubes was added and the mixture was further stir-fried for 5 minutes. Thereafter, 5.8 mass % of oatmeal and 3.5 mass % of breadcrumbs were added and stir-fried for 2 minutes, followed by the addition of 0.4 mass % of salt and pepper, and then the mixture was molded using a cylindrical mold with a diameter of 4 cm and a height of 3 cm to obtain a composition of Comparative Example 15. The oatmeal used was “Topvalu Instant Oatmeal: Mild Flavor and Easy to Eat,” available from Aeon Co., Ltd.

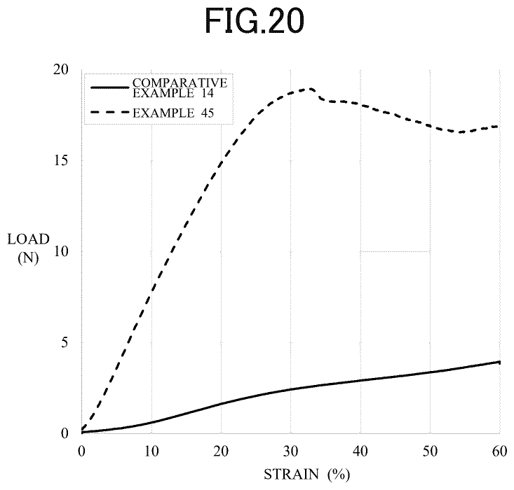

For both of the compositions of Example 45 and Comparative Example 14, an appropriate amount of oil was added to a frying pan, heated to 180° C. as a finishing step, and the content of poorly soluble β-glucan, the moldability evaluation based on a load test, and the sensory evaluation were performed in the same manner as described above. The obtained results are shown in Tables 57 to 59 and FIG. 20.

TABLE 57

| | Structuring | Comparative | |
|---|---|---|---|
| | material | Example 14 | Example 45 |
| Eringi | — | 70.0 | 57.4 |
| Oil | — | 5.3 | 5.3 |
| Onion | — | 15.0 | 15.0 |
| Oatmeal | — | 5.8 | 5.8 |
| Breadcrumbs | — | 3.5 | 3.5 |
| Salt and pepper | — | 0.4 | 0.0 |
| Transglutaminase | ◯ (93%) | 0.0 | 7.0 |
| preparation A | | | |
| Pea protein | ◯ | 0.0 | 6.0 |

| | | |
|---|---|---|
| Total | 100.0 | 100.0 |
| Mushroom solid content | 7.0 | 5.7 |
| Dietary fiber | 2.4 | 2.0 |
| Poorly soluble β-glucan | 1.39 | 1.13 |
| Structuring material | 0.0 | 12.5 |

(Unit of numerical values: mass %)

TABLE 58

| | Comparative | |
|---|---|---|
| | Example 14 | Example 45 |
| Presence or absence of peak | Absent | Present |
| Load at 30% strain (N) | 2.4 | 18.7 |

TABLE 59

| | Comparative | |
|---|---|---|
| | Example 14 | Example 45 |
| Hardness | 1.3 | 3.7 |
| Chewiness | 1.7 | 3.3 |
| Tastiness | 2.0 | 3.5 |
| Total score | 5.0 | 10.5 |
| Determination | Fail | Pass |

### (Type of Non-Mushroom-Derived Protein Material)

Each food composition of Examples 46 to 49 was produced in the same manner as in Example 1 using various protein materials according to the formulations shown in Table 60. The following protein materials were used.
Pea: “Pea Protein Derived from Peas” (Usuki Pharmaceutical Co., Ltd.)
Milk protein: “Whey Protein W80, Plain Flavor” (Nippon Garlic Corporation)
Defatted soybeans: “Showa Fresh RF” (Showa Sangyo Co., Ltd.)
Sodium caseinate: “Sodium Caseinate” (FUJIFILM Wako Pure Chemical Corporation)

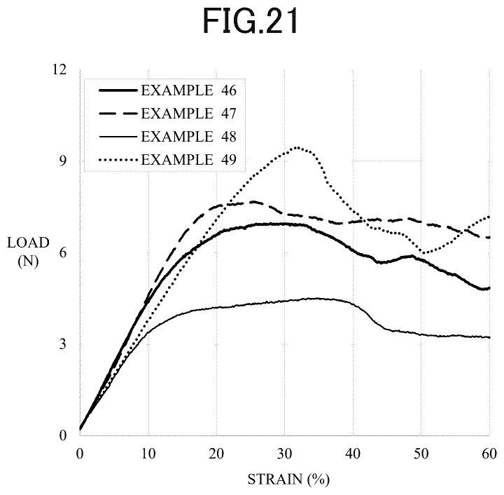

For each of the food compositions of Examples 46 to 49, the content of poorly soluble β-glucan, the moldability evaluation based on a load test, and the sensory evaluation were performed in the same manner as described above. The obtained results are shown in Tables 60 to 62 and FIG. 21.

TABLE 60

| | Structuring | | | | |
|---|---|---|---|---|---|
| | material | Example 46 | Example 47 | Example 48 | Example 49 |
| Protein type | | Pea | Milk protein | Defatted soybeans | Sodium caseinate |
| Pressed eringi | — | 80.0 | 80.0 | 80.0 | 80.0 |
| Transglutaminase preparation B | O(8%) | 1.0 | 1.0 | 1.0 | 1.0 |
| Dextrin | — | 9.0 | 9.0 | 9.0 | 9.0 |
| Each protein material | ◯ | 10.0 | 10.0 | 10.0 | 10.0 |

| | | | | |
|---|---|---|---|---|
| Total | 100.0 | 100.0 | 100.0 | 100.0 |
| Mushroom solid content | 13.6 | 13.6 | 13.6 | 13.6 |
| Dietary fiber | 5.4 | 5.4 | 5.4 | 5.4 |
| Poorly soluble β-glucan | 1.69 | 1.69 | 1.69 | 1.69 |
| Structuring material | 10.1 | 10.1 | 10.1 | 10.1 |

(Unit of numerical values: mass %)

TABLE 61

| | Example 46 | Example 47 | Example 48 | Example 49 |
|---|---|---|---|---|
| Presence or absence of peak | Present | Present | Present | Present |
| Load at 30% strain (N) | 7.0 | 7.3 | 4.4 | 9.3 |

TABLE 62

| | Example 46 | Example 47 | Example 48 | Example 49 |
|---|---|---|---|---|
| Hardness | 3.7 | 3.7 | 2.7 | 4.2 |
| Chewiness | 2.3 | 2.5 | 2.2 | 2.5 |
| Tastiness | 3.0 | 3.8 | 2.7 | 3.5 |
| Total score | 9.0 | 10.0 | 7.6 | 10.2 |
| Determination | Pass | Pass | Pass | Pass |

### (Polysaccharide-Processed Products Using Various Mushrooms)

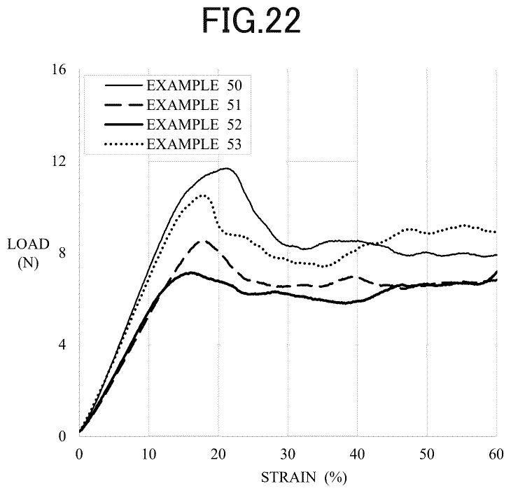

Examples 50 to 53 were molded and structured in the same manner as in Example 12 according to the formulations shown in Table 63. For each of the food compositions of Examples 50 to 53, the content of poorly soluble β-glucan, the moldability evaluation based on a load test, and the sensory evaluation were performed in the same manner as described above. The obtained results are shown in Tables 63 to 65 and FIG. 22.

TABLE 63

| | Structuring | | | | |
|---|---|---|---|---|---|
| Details | material | Example 50 | Example 51 | Example 52 | Example 53 |
| Eringi | — | 50.0 | 0.0 | 0.0 | 0.0 |
| Enokitake | — | 0.0 | 50.0 | 0.0 | 0.0 |
| Oyster mushroom | — | 0.0 | 0.0 | 50.0 | 0.0 |
| Buna-shimeji | — | 0.0 | 0.0 | 0.0 | 50.0 |
| Sodium alginate preparation | ◯ (63%) | 4.6 | 4.6 | 4.6 | 4.6 |
| Dextrin | — | 14.4 | 14.4 | 14.4 | 14.4 |
| Breadcrumbs | — | 10.0 | 10.0 | 10.0 | 10.0 |
| Water | — | 20.0 | 20.0 | 20.0 | 20.0 |
| Salt and pepper | — | 1.0 | 1.0 | 1.0 | 1.0 |

| | | | | |
|---|---|---|---|---|
| Total | 100.0 | 100.0 | 100.0 | 100.0 |
| Mushroom solid content | 5.0 | 5.7 | 5.3 | 4.5 |
| Dietary fiber | 1.7 | 1.7 | 1.7 | 1.7 |
| Poorly soluble β-glucan | 0.99 | 0.30 | 0.60 | 0.52 |
| Structuring material | 2.9 | 2.9 | 2.9 | 2.9 |

(Unit of numerical values: mass %)

TABLE 64

| | Example 50 | Example 51 | Example 52 | Example 53 |
|---|---|---|---|---|
| Presence or absence of peak | Present | Present | Present | Present |
| Load at 30% strain (N) | 8.3 | 6.5 | 6.2 | 7.7 |

TABLE 65

| | Example 50 | Example 51 | Example 52 | Example 53 |
|---|---|---|---|---|
| Hardness | 4.0 | 3.5 | 3.3 | 3.7 |
| Chewiness | 3.0 | 2.8 | 2.8 | 3.0 |
| Tastiness | 3.5 | 3.5 | 3.3 | 3.2 |
| Total score | 10.5 | 9.8 | 9.4 | 9.9 |
| Determination | Pass | Pass | Pass | Pass |

### (Presence or Absence of Standing Step)

In the formulations shown in Table 66, molding was performed in the same manner as in Example 1, and food compositions were prepared by allowing them to stand under different temperature conditions. After molding, Comparative Example 15 and Examples 54 and 55 were allowed to stand for 0, 3, and 48 hours, respectively, in a refrigerator set at 10° C. or lower. Examples 56 and 57 were wrapped in plastic wrap and allowed to stand for 1 or 3 hours in a dryer (DKM600, Yamato Scientific Co., Ltd.) set at 50° C. In order to stop the structuring reaction, steaming was performed for 15 minutes in a covered pot to which a small amount of water had been added.

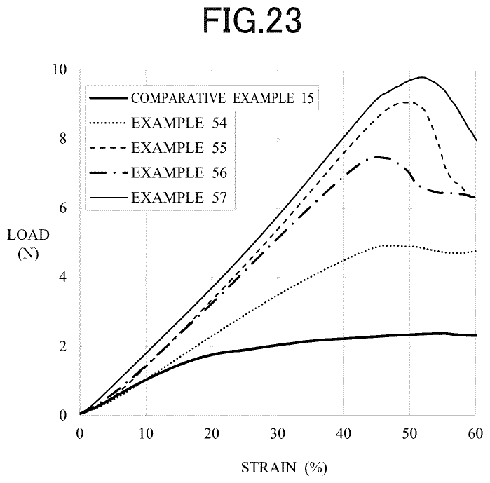

For each of the food compositions of Comparative Example 15 and Examples 54 to 57, the content of poorly soluble β-glucan, the moldability evaluation based on a load test, and the sensory evaluation were performed in the same manner as described above. The obtained results are shown in Tables 66 to 68 and FIG. 23.

TABLE 66

| | | | | | |
|---|---|---|---|---|---|
| Sructuring | Comparative | | | | |
| material | Example 15 | Example 54 | Example 55 | Example 56 | Example 57 |

| | | | | | |
|---|---|---|---|---|---|
| Standing time | 0 h | 4° C. 3 h | 4° C. 48 h | 50° C. 1 h | 50° C. 3 h |

| | | |
|---|---|---|
| Eringi | — | 50.0 |
| Transglutaminase preparation A | ◯ (93%) | 6.0 |
| Breadcrumbs | — | 13.6 |
| Sugar | — | 1.3 |
| Salt | — | 1.0 |
| White pepper | — | 0.1 |
| Soy protein | ◯ | 5.0 |
| Dextrin | — | 3.0 |
| Onion | — | 20.0 |

| | |
|---|---|
| Total | 100.0 |
| Mushroom solid content | 5.0 |
| Dietary fiber | 1.7 |
| Poorly soluble β-glucan | 0.99 |
| Structuring material | 10.6 |

(Unit of numerical values: mass %)

TABLE 67

| | Comparative | | | | |
|---|---|---|---|---|---|
| | Example 15 | Example 54 | Example 55 | Example 56 | Example 57 |
| Presence or absence of peak | Absent | Present | Present | Present | Present |
| Load at 30% strain (N) | 2.0 | 3.5 | 5.4 | 5.1 | 5.8 |

TABLE 68

| | Comparative | | | | |
|---|---|---|---|---|---|
| | Example 15 | Example 54 | Example 55 | Example 56 | Example 57 |
| Hardness | 1.5 | 2.8 | 4.2 | 4.2 | 4.3 |
| Chewiness | 1.5 | 2.3 | 3.3 | 3.0 | 3.5 |
| Tastiness | 2.5 | 3.5 | 4.5 | 4.5 | 4.3 |
| Total score | 5.5 | 8.6 | 12.0 | 11.7 | 12.1 |
| Determination | Fail | Pass | Pass | Pass | Pass |

1. A food composition comprising:
mushroom-derived solid content; and
a structuring material that structures the solid content together, wherein
the solid content in the food composition is 1.3 to 49.0 mass %,
the content of the structuring material in the food composition is 1.5 to 90.0 mass %, and
the food composition has a peak at a strain of 10% to 60% and a load of 2 N or more at a strain of 30% in the following load test,
(Load test)
a cylindrical plunger having a diameter of 1 cm is attached to a rheometer, and the load of a test sample (height: 3 cm) is measured at a speed of 1 mm/s at room temperature.

2. The food composition according to claim 1, wherein the food composition contains 0.4 to 25.0 mass % of dietary fiber.

3. The food composition according to claim 2, wherein the food composition contains 0.05 to 8.0 mass % of mushroom-derived poorly soluble β-glucan.

4. The food composition according to claim 1, wherein the structuring material is selected from the group consisting of non-mushroom-derived protein materials, protein crosslinking enzymes, alginic acids, and mannans.

5. The food composition according to claim 2, wherein the structuring material is selected from the group consisting of non-mushroom-derived protein materials, protein crosslinking enzymes, alginic acids, and mannans.

6. The food composition according to claim 4, wherein the non-mushroom-derived protein materials include one or more selected from the group consisting of beans, cereals, natural meats such as livestock meat, and dairy products.

7. The food composition according to claim 4, wherein the protein crosslinking enzymes include transglutaminase.

8. The food composition according to claim 5, wherein the protein crosslinking enzymes include transglutaminase.

9. The food composition according to claim 4, wherein the alginic acids are selected from the group consisting of alginic acid, sodium alginate, potassium alginate, calcium alginate, alginate esters, and ammonium alginate.

10. The food composition according to claim 4, wherein the mannans are selected from the group consisting of konjac powder, konjac mannan, and glucomannan.

11. The food composition according to claim 1, wherein the mushrooms are fruiting bodies.

12. The food composition according to claim 1, wherein the mushrooms are one or more selected from the group consisting of enokitake (Flammulina velutipes), eringi (Pleurotus eryngii), wood ear mushroom (Auricularia auricula-judae), shiitake (Lentinula edodes), button mushroom (Agaricus bisporus), nameko (Pholiota nameko), oyster mushroom (Pleurotus ostreatus), buna-shimeji (Hypsizygus marmoreus), and maitake (Grifola frondosa).

13. The food composition according to claim 2, wherein the mushrooms are one or more selected from the group consisting of enokitake (Flammulina velutipes), eringi (Pleurotus eryngii), wood ear mushroom (Auricularia auricula-judae), shiitake (Lentinula edodes), button mushroom (Agaricus bisporus), nameko (Pholiota nameko), oyster mushroom (Pleurotus ostreatus), buna-shimeji (Hypsizygus marmoreus), and maitake (Grifola frondosa).

14. A dry food composition obtained by drying the food composition according to claim 1.

15. A method for producing a food composition, comprising:
a mixing step of mixing mushroom-derived solid content with a structuring material that structures the solid content together; and
a standing step of allowing the mixture obtained by the mixing step to stand under an environment of 3° C. to 60° C. for 1.0 to 48 hours, wherein
the solid content in the food composition is 1.3 to 49.0 mass %, and
the content of the structuring material in the food composition is 1.5 to 90.0 mass %.

16. The method for producing a food composition according to claim 15, wherein the food composition contains 0.4 to 25.0 mass % of dietary fiber.

17. The method for producing a food composition according to claim 15, wherein the food composition contains 0.05 to 8.0 mass % of mushroom-derived poorly soluble β-glucan.

18. The method for producing a food composition according to claim 15, wherein the structuring material is selected from the group consisting of non-mushroom-derived protein materials, protein crosslinking enzymes, alginic acids, and mannans.

19. The method for producing a food composition according to claim 16, wherein the structuring material is selected from the group consisting of non-mushroom-derived protein materials, protein crosslinking enzymes, alginic acids, and mannans.
